SEQUENCE LISTING (4 sequences)

SEQ ID NO: 1 - protein, 14 aa, synthetic construct
KDDYAGGGAAGKDA

SEQ ID NO: 2 - protein, 10 aa, synthetic construct
GKAGEAGYRA

SEQ ID NO: 3 - protein, 11 aa, synthetic construct
GAKAGEAGYRA

SEQ ID NO: 4 - protein, 9 aa, synthetic construct
  SITE 8: note = Propargylglycine
  MOD_RES 9: note = Amidation of carboxyl
AKGAGRYXN

(19) United States
(12) Patent Application Publication
(10) Pub. No.: US 2025/0035638 A1
(43) Pub. Date: Jan. 30, 2025

(54) METHODS AND SYSTEMS FOR AUTOMATED SAMPLE PROCESSING

(71) Applicant: Erisyon Inc., Austin, TX (US)

(72) Inventors: Sasson Somekh, Los Altos, CA (US); Robert Lowrance, Morgan Hill, CA (US); Yevgeniy Rabinovich, Fremont, CA (US); Emily Babcock, Austin, TX (US)

(21) Appl. No.: 18/710,558

(22) PCT Filed: Nov. 16, 2022
(86) PCT No.: PCT/US2022/079979

§ 371 Date: May 15, 2024

Related U.S. Application Data
(60) Provisional application No. 63/280,291, filed on Nov. 17, 2021.

(51) Int. Cl. G01N 33/68; G01N 33/58; G01N 35/00

(52) U.S. Cl. CPC G01N 33/6818; G01N 33/582; G01N 35/0099

(57) ABSTRACT

A method for processing a biomolecule may comprise providing, at a first location, (i) a substrate and (ii) a substrate holder coupled to the substrate. The substrate may comprise the biomolecule coupled thereto. The substrate holder and the substrate may be automatically directed from the first location to a second location different from the first location. At the second location, the biomolecule may be processed to provide a processed biomolecule coupled to the substrate. The processing may comprise labelling the biomolecule to provide a labeled biomolecule.

### CROSS-REFERENCE

This application is a U.S. 371 national phase application of International Application No. PCT/US2022/079979, filed Nov. 16, 2022, which claims the benefit of and priority to U.S. Application No. 63/280,291, filed Nov. 17, 2021, the contents of each of which are incorporated by reference herein in their entirety.

### SEQUENCE LISTING

This application contains a Sequence Listing which has been submitted electronically in XML format. The Sequence Listing XML is incorporated herein by reference. Said XML file, created on May 6, 2024, is named ERY-018WOUS_SL.xml and is 4.98 kilobytes in size.

### BACKGROUND

Fluorosequencing techniques can be used for a variety of different biomolecule identification and sequencing methodologies. Such fluorosequencing techniques can use preparatory methods to prepare a sample for fluorosequencing, and such methods can have a number of operations to complete before the sample is ready.

### SUMMARY

In an aspect, the present disclosure provides methods and systems for processing biomolecules using automated handling systems. Such automated systems can offer numerous improvements over non-automated sample processing, including the ability to monitor and control various conditions of the processing (e.g., temperature, pressure, light intensity, chemical exposure, time) that may not be monitored in non-automated systems. For example, a user performing a sample processing method may not have the ability to tightly control reaction time and light exposure, while an automated system can control such parameters. The automated system may also improve efficiency over a user performing a non-automated process. For example, an automated system can tightly control process operations to increase reaction yields and may be less susceptible to spill errors and the like. The automated system can improve throughput (e.g., an automated system can process multiple samples at once, while a user may be able to process a single sample). Additionally, automated system can enable error management strategies, which can reduce long term errors and further improve efficiency.

In an aspect, the present disclosure provides a method for processing a biomolecule, comprising: (a) providing, at a first location, (i) a substrate and (ii) a substrate holder coupled to the substrate, wherein the substrate comprises the biomolecule coupled thereto; (b) automatically directing the substrate holder and the substrate from the first location to a second location different from the first location; and (c) at the second location, processing the biomolecule to provide a processed biomolecule coupled to the substrate; wherein the processing comprises labelling the biomolecule to provide a labelled biomolecule.

In some embodiments, the method for processing the biomolecule is completely automated by a computer processor. In some embodiments, the substrate is coupled to the substrate holder at a first position of the substrate holder, and the substrate holder is coupled to an additional substrate comprising an additional biomolecule at a second position of the substrate holder. In some embodiments, the substrate is coupled to the substrate holder at a first position of the substrate holder, and the substrate holder is coupled to a plurality of additional substrates comprising a plurality of additional biomolecules at a plurality of positions of the substrate holder. In some embodiments, the plurality of substrates comprises at least 8 substrates. In some embodiments, the method further comprises, coupling the biomolecule to the substrate. In some embodiments, the coupling occurs at an additional location different from the first location or the second location. In some embodiments, the coupling occurs at the first location. In some embodiments, the biomolecule comprises a protein or a peptide. In some embodiments, the substrate is a bead or a lantern. In some embodiments, the substrate is a solid support. In some embodiments, (b) is performed using a robotic system configured to direct the substrate holder from the first location to the second location. In some embodiments, the robotic system further comprises a fluid handling unit, a moveable stage, an evaporation unit, a vacuum, a gas pump, a light source, a temperature control unit, a shaker, a fan, or a combination thereof. In some embodiments, the robotic system comprises the fluid handling unit, and the method further comprises, prior to (b), providing a well and using the fluid handling unit to provide reagents into the well. In some embodiments, the reagents comprise labelling agents. In some embodiments, the labelling agents are fluorescent labelling agents. In some embodiments, the labelling agents are configured to label one or more amino acids of a peptide. In some embodiments, the labelling agents comprise a labelling agent configured to label a lysine residue, cysteine residue, glutamic acid residue, aspartic acid residue, tyrosine residue, arginine residue, histidine residue, threonine residue, serine residue, proline residue, glutamine residue, or tryptophan residue. In some embodiments, the labelling agents are configured to label post-translational modifications such as phosphorylation, glycosylation, ubiquitination, or methylation. In some embodiments, the processing of (c) comprises contacting the substrate coupled to the biomolecule with the reagents in the well to provide the labelled biomolecule. In some embodiments, the robotic system further comprises the shaker, and (c) further comprises, using the shaker to mix the reagents in the well. In some embodiments, the robotic system further comprises the light source. In some embodiments, (c) further comprises using the light source to attach one or more reagents of the reagents to the biomolecule, thereby providing the labelled biomolecule. In some embodiments, the robotic system comprises, the moveable stage, and the method further comprises, using the moveable stage to move the well relative to the fluid handling unit. In some embodiments, the robotic system comprises the evaporation unit, wherein the processing of (c) comprises use of a solvent, and the method further comprises, subsequent to (c), using the evaporation unit to evaporate the solvent from the labelled biomolecule. In some embodiments, the evaporation unit comprises the vacuum. In some embodiments, the evaporation unit comprises the gas pump. In some embodiments, the gas pump is coupled to a nitrogen gas stream. In some embodiments, the robotic system comprises the temperature control unit, and where (c) is performed at a controlled temperature. In some embodiments, the robotic system comprises a feedback mechanism that regulates the fluid handling unit, the moveable stage, the evaporation unit, the vacuum, the gas pump, the light source, the temperature control unit, the shaker, the fan, or the combination thereof. In some embodiments, the robotic system accommodates a multi well plate. In some embodiments, the method further comprises, providing a light source at the second location. In some embodiments, the light source is a light emitting diode (LED) light source. In some embodiments, the light source is configured to illuminate in a pre-specified pattern. In some embodiments, the light source is configured to couple to a multiwell plate and illuminate one or more wells of the multiwell plate. In some embodiments, (c) comprises using the light source to conduct a photoreaction to provide the labelled biomolecule. In some embodiments, the photoreaction comprises decarboxylative alkylation. In some embodiments, the biomolecule comprises a peptide, and the labelling comprises coupling a label to an amino acid of the peptide. In some embodiments, the label comprises (i) a first reactive group that is configured to couple to a second reactive group that is coupled to a reporter moiety configured to emit a signal or (ii) a protecting group configured to prevent coupling between the label and the second reactive group. In some embodiments, the method further comprises, removing the labelled biomolecule from the substrate. In some embodiments, the removing comprises contacting the substrate with an acidic buffer (e.g., with a pH of 1 to 4, such as formic acid, trifluoroacetic acid (TFA), or glycine hydrochloride). In some embodiments, the method further comprises, using an evaporation unit to remove the acidic buffer (e.g., TFA) from the labelled biomolecule. In some embodiments, the method further comprises, storing the labelled biomolecule. In some embodiments, the method further comprises, evaporating the removed biomolecule. In some embodiments, the evaporating is performed using an evaporation unit. In some embodiments, the evaporation unit is coupled to a gas source or a vacuum. In some embodiments, the gas source is a nitrogen stream. In some embodiments, the method further comprises, subjecting the labelled biomolecule to sequencing. In some embodiments, the labelled biomolecule is a labelled peptide or protein and the sequencing comprises protein sequencing. In some embodiments, the protein sequencing comprises fluorosequencing. In some embodiments, the method further comprises, prior to the sequencing, coupling the labelled biomolecule to a flow cell. In some embodiments, the flow cell comprises a linker configured to couple to the labelled biomolecule. In some embodiments, the flow cell comprises a set of linkers, each of the set of linkers configured to couple to one of a set of biomolecules. In some embodiments, the substrate comprises a peptide capture reagent. In some embodiments, the peptide capture reagent comprises a cleavable linker. In some embodiments, the peptide capture reagent comprises an N-terminal capture reagent. In some embodiments, the N-terminal capture reagent is pyridinecarbaldehyde (PCA) or a derivative thereof. In some embodiments, the substrate comprises a poly-ethylene glycol (PEG) linker, polyacrylate, polyamide, or polystyrene. In some embodiments, the biomolecule is a protein, a peptide, a lipid, a carbohydrate, a metabolite, a nucleic acid molecule, or a combination thereof. In some embodiments, the biomolecule is obtained from a biological sample. In some embodiments, the biological sample is a cell or tissue sample. In some embodiments, the biological sample is a blood sample.

In another aspect, the present disclosure provides a system for processing a biomolecule, comprising: a substrate comprising the biomolecule coupled thereto; a substrate holder configured to couple to the substrate; an automated robotic handling system configured to automatically direct the substrate holder from a first location to a second location; and reagents for labelling the biomolecule.

In some embodiments, the substrate is a bead or a lantern. In some embodiments, the substrate is coupled to the substrate holder at a first position of the substrate holder, and the substrate holder is coupled to an additional substrate comprising an additional biomolecule at a second position of the substrate holder. In some embodiments, the system further comprises, a light source at the second location. In some embodiments, the light source is an LED light source. In some embodiments, the light source is configured to illuminate in a pre-specified pattern. In some embodiments, the light source is configured to couple to a multiwell plate and illuminate one or more wells of the multi well plate. In some embodiments, the substrate is coupled to the substrate holder at a first position of the substrate holder, and the substrate holder is coupled to a plurality of additional substrates comprising a plurality of additional biomolecules at a plurality of positions of the substrate holder. In some embodiments, the plurality of substrates comprises at least 8 substrates. In some embodiments, the system further comprises a robotic system configured to direct the substrate holder from a first location to a second location. In some embodiments, the robotic system further comprises a fluid handling unit, a moveable stage, an evaporation unit, a vacuum, a gas pump, a light source, a temperature control unit, a shaker, a fan, or a combination thereof. In some embodiments, the robotic system comprises a feedback mechanism that is configured to regulate the fluid handling unit, the moveable stage, the evaporation unit, the vacuum, the gas pump, the light source, the temperature control unit, the shaker, the fan, or the combination thereof. In some embodiments, the robotic system comprises an evaporation unit configured to evaporate a solvent. In some embodiments, the evaporation unit comprises a vacuum. In some embodiments, the evaporation unit comprises a gas pump. In some embodiments, the gas pump is coupled to a nitrogen gas stream. In some embodiments, the robotic system is configured to accommodate a multi well plate. In some embodiments, the reagents for labelling comprise labelling agents configured to label one or more amino acids of a peptide. In some embodiments, the labelling agents comprise a labelling agent configured to label a lysine residue, cysteine residue, glutamic acid residue, aspartic acid residue, tyrosine residue, arginine residue, histidine residue, threonine residue, serine residue, proline residue, glutamine residue, or tryptophan residue. In some embodiments, the labelling agents are configured to label post-translational modifications such as phosphorylation, glycosylation, ubiquitination, or methylation. In some embodiments, the labelling agents comprise agents for performing click chemistry. In some embodiments, the labelling agents comprise (i) a first reactive group that is configured to couple to a second reactive group that is coupled to a reporter moiety configured to emit a signal or (ii) a protecting group configured to prevent coupling between the label and the second reactive group. In some embodiments, the biomolecule comprises a peptide. In some embodiments, the system further comprises reagents for removing the labelled biomolecule from the substrate. In some embodiments, the reagents comprise an acidic buffer (e.g., with a pH of 1 to 4, such as formic acid, trifluoroacetic acid (TFA), or glycine hydrochloride). In some embodiments, the system further comprises, a storage unit for storing the labelled biomolecule. In some embodiments, the system further comprises, a flow cell configured to couple to a labelled biomolecule. In some embodiments, the labelled biomolecule is a labelled peptide or protein. In some embodiments, the flow cell comprises a linker configured to couple to the labelled biomolecule. In some embodiments, the flow cell comprises a set of linkers, each of the set of linkers configured to couple to one of a set of biomolecules. In some embodiments, the substrate comprises a peptide capture reagent. In some embodiments, the peptide capture reagent comprises a cleavable linker. In some embodiments, the peptide capture reagent comprises an N-terminal capture reagent. In some embodiments, the N-terminal capture reagent is pyridinecarbaldehyde (PCA) or a derivative thereof. In some embodiments, the substrate comprises a poly-ethylene glycol (PEG) linker, polyacrylate, polyamide, or polystyrene. In some embodiments, the biomolecule is obtained from a biological sample. In some embodiments, the biological sample is a cell or tissue sample. In some embodiments, the biological sample is a blood sample.

In another aspect, the present disclosure provides a system for processing a biomolecule, comprising: a substrate holder configured to couple to a substrate comprising the biomolecule coupled thereto; a robotic arm coupled to the substrate holder; at least one computer processor configured to perform executable instructions and a memory comprising the executable instructions, which, when executed by the at least one computer processor, causes the at least one computer processor to implement a method comprising: automatically instructing the robotic arm to direct the substrate holder and the substrate from a first location to a second location different from the first location, wherein, at the second location, the biomolecule is labelled to provide a labelled biomolecule coupled to the substrate.

In some embodiments, the system further comprises a fluid handling unit. In some embodiments, the method further comprises instructing the fluid handling unit to provide reagents in a vessel in the second location. In some embodiments, the reagents comprise labelling agents. In some embodiments, the system further comprises a moveable stage. In some embodiments, the method further comprises instructing the moveable stage to move from the first location to the second location or from the second location to a third location different from the second location. In some embodiments, the system further comprises an evaporation unit. In some embodiments, the method further comprises instructing the robotic arm to move the evaporation unit. In some embodiments, the evaporation unit is coupled to a vacuum or a gas pump. In some embodiments, the system further comprises a light source. In some embodiments, the method further comprises electronically turning on or off the light source or modulating an intensity thereof. In some embodiments, the system further comprises a temperature control unit. In some embodiments, the method further comprises, controlling the temperature control unit to obtain a temperature within a range of temperatures. In some embodiments, the temperature control unit comprises a fan or Peltier component.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an automated robotic handling system.

FIG. 2 shows a layout of components for an automated robotic handling system.

FIG. 3A provides a sectional view of a lantern bar.

FIG. 3B provides a full view of a lantern bar.

FIG. 4 illustrates a shaker element.

FIG. 5 illustrates a combined heating and evaporation element.

FIG. 6 illustrates a detail of an evaporation bar.

FIG. 7 illustrates an output stage.

FIG. 8 illustrates a well plate.

FIG. 9 shows a computer system that is programmed or otherwise configured to implement methods provided herein.

FIG. 10 schematically illustrates an example of a method 1000 for processing a biomolecule.

FIG. 11A provides an exploded view of a hermetically sealed lantern or N2 bar system.

FIG. 11B provides a sectional view of a hermetically sealed lantern or N2 bar.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may refer to within an acceptable error range for the particular value, which may depend in part on how the value is measured or determined, e.g., the limitations of the measurement system. For example, “about” may refer to within 1 or more than 1 standard deviation. Alternatively, “about” may refer to a range of up to 20%, up to 10%, up to 5%, or up to 1% of a given value. Where particular values are described in the application and claims, unless otherwise stated the term “about” meaning within an acceptable error range for the particular value may be assumed.

The term “analyte” or “analytes,” as used herein, generally refers to a molecule whose presence or absence is measured or identified. An analyte can be a molecule for which a detectable probe or assay exists or can be produced. For example, an analyte can be a macromolecule, such as, for example, a nucleic acid, a polypeptide, a carbohydrate, a small organic, an inorganic compound, or an element, for example, gold, iron, or lead. An analyte can be part of a sample that contains other components, or can be the sole or the major component of the sample. An analyte can be a component of a whole cell or tissue, a cell or tissue extract, a fractionated lysate thereof or a substantially purified molecule. In some embodiments, the target analyte is a polypeptide.

The terms “polypeptide” and “peptide” generally to refer to a polymer of amino acids in which an amino acid may be linked to another amino acid by a peptide bond. In some examples, a polypeptide is a protein. The amino acid may be a naturally occurring amino acid or a non-naturally occurring amino acid (e.g., amino acid analogue). The polymer can be linear or branched and can include modified amino acids, and/or may be interrupted by non-amino acids. Polypeptides can occur as single chains or associated chains. The polymer may include a plurality of amino acids and may have a secondary and tertiary structure (e.g., protein). In some examples, the polymer comprises at least 2, 3, 4, 5, 6, 7, 8, 9, 10, 20, 30, 40, 50, 100, 1000, 10,000, or more amino acids.

The term “amino acid,” as used herein, generally refers to a naturally occurring or non-naturally occurring amino acid (amino acid analogue). The non-naturally occurring amino acid may be a synthesized amino acid. As used herein, the terms “amino acid sequence,” “peptide sequence,” and “polypeptide sequence,” as used herein, generally refer to at least two amino acids or amino acid analogs that are covalently linked by a peptide (amide) bond or an analog of a peptide bond. The term peptide includes oligomers and polymers of amino acids or amino acid analogs. The amino acids of the peptide may be L-amino acids or D-amino acids. A peptide, polypeptide, or protein may be synthetic, recombinant, or naturally occurring. A synthetic peptide may be a peptide that is produced by artificial approaches in vitro.

As used herein, the term “side chains” or “R” generally refers to unique structures attached to the alpha carbon (attaching the amine and carboxylic acid groups of the amino acid) that render uniqueness to each type of amino acid. R groups have a variety of shapes, sizes, charges, and reactivities, such as charged polar side chains, either positively or negatively charged, such as lysine (+), arginine (+), histidine (+), aspartate (−), and glutamate (−); amino acids can also be basic, such as lysine, or acidic, such as glutamic acid; uncharged polar side chains have hydroxyl, amide, or thiol groups, such as cysteine having a chemically reactive side chain, e.g., a thiol group that can form bonds with another cysteine, serine (Ser) and threonine (Thr), that have hydroxylic R side chains of different sizes; asparagine (Asn), glutamine (Gin), and tyrosine (Tyr); non-polar hydrophobic amino acid side chains include the amino acid glycine, alanine, valine, leucine, and isoleucine having aliphatic hydrocarbon side chains ranging in size from a methyl group for alanine to isomeric butyl groups for leucine and isoleucine; methionine (Met) has a thiol ether side chain; proline (Pro) has a cyclic pyrrolidine side group. Phenylalanine (with its phenyl moiety) (Phe) and tryptophan (Trp) (with its indole group) contain aromatic side chains, which are characterized by bulk as well as lack of polarity.

The term “cleavable unit,” as used herein, generally refers to a molecule that can be split into at least two molecules. Non-limiting examples of cleavage reagents and conditions to split a cleavable unit include: enzymes, nucleophilic or basic reagents, reducing agents, photo-irradiation, electrophilic or acidic reagents, organometallic or metal reagents, and oxidizing reagents.

The term “sample,” as used herein, generally refers to a sample containing or suspected of containing a polypeptide. For example, a sample can be a biological sample containing one or more polypeptides. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and food samples (e.g., dairy products, vegetable products, and meat products). The sample may be processed prior to loading into a microfluidic device. For example, the sample may be processed to purify the polypeptides and/or to include reagents.

As used herein, sequencing of peptides “at the single molecule level” generally refers to amino acid sequence information obtained from individual (e.g., single) peptide molecules in a mixture of diverse peptide molecules. The amino acid sequence information may be obtained from an entirety of an individual peptide molecule or one or more portion of the individual peptide molecule, such as a contiguous amino acid sequence of at least a portion of the individual peptide molecule. Alternatively, partial amino acid sequence information may be obtained, which may allow for identification of the peptide or protein. Partial amino acid sequence information, including for example, the pattern of a specific amino acid residue (e.g., lysine) within individual peptide molecules, may be sufficient to uniquely identify an individual peptide molecule. For example, a pattern of amino acids may comprise a plurality of identified positions (e.g., identified as a particular amino acid type, such as lysine, or identified as a particular set of amino acids, such as the set of carboxylate side chain-containing amino acids), and a plurality of unidentified positions. The sequence of identified positions may be searched against a proteome (e.g., database) of a given organism to identify the individual peptide molecule. In some examples, sequencing of a peptide at the single molecule level may identify a pattern of a certain type of amino acid (e.g., lysine) in an individual peptide molecule. Such information may be used to identify a macromolecule (e.g., protein) from which the peptide was derived. This may advantageously preclude the need to identify all amino acids of the peptide.

As used herein, the term “Edman degradation” generally refers to methods comprising chemical removal of amino acids from peptides or proteins. In some cases, Edman degradation denotes terminal (e.g., N- or C-terminal) amino acid removal. In specific cases, Edman degradation refers to N-terminal amino acid removal through isothiocyanate (e.g., phenyl isothiocyanate) coupling and cyclization with the terminal amine group of an N-terminal residue, such that the N-terminal amino acid is removed from a peptide. In some cases, Edman degradation broadly encompasses N-terminal amino acid functionalization leading to N-terminal amino acid removal. In some cases, Edman degradation encompasses C-terminal amino acid removal. In some cases, Edman degradation comprises terminal amino acid functionalization (e.g., N-terminal amino acid isothiocyanate functionalization) followed by enzymatic removal (e.g., by an ‘Edmanase’ with specificity for chemically derivatized N-terminal amino acids).

As used herein, the term “single molecule sensitivity” generally refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). This may include the ability to simultaneously record the fluorescent intensity of multiple individual (e.g., single) peptide molecules distributed across the glass surface. Optical devices are commercially available that can be applied in this manner. For example, a microscope equipped with total internal reflection illumination and an intensified charge-couple device (CCD) or complementary metal-oxide semiconductor (CMOS) detector is available. Imaging with a high sensitivity CCD or CMOS camera allows the instrument to simultaneously record the fluorescent intensity of multiple individual (e.g., single) peptide molecules distributed across a surface. Image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment. The methods and systems described herein may be configured to prepare a sample for, or perform a data acquisition with, single molecule sensitivity sensitive measurements.

As used herein, the term “support” generally refers to an entity to which a substance (e.g., molecular construct) can be immobilized. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, or an aldehyde dithiolane. The functional group may couple specifically to an N-terminus or a C-terminus of a peptide. The functional group may couple specifically to an amino acid side chain. The functional group may couple to a side chain of an amino acid (e.g., the acid of a glutamate or aspartate, the thiol of a cysteine, the amine of a lysine, or the amide of a glutamine, or asparagine). The functional group may couple specifically to a reactive group on a particular species, such as a label. In some examples of functionalized beads, the functional motif can be reversibly coupled and cleaved. A functional motif can also irreversibly couple to a molecule.

As used herein, the term “array” generally refers to a population of sites. Such populations of sites can be differentiated from one another according to relative location. Different molecules that are at different sites of an array can be differentiated from each other according to the locations of the sites in the array. An individual site of an array can include one or more molecules of a particular type. For example, a site can include a single polypeptide having a particular sequence or a site can include several polypeptides having the same sequence. The sites of an array can be different features located on the same substrate. Such features may include, without limitation, wells in a substrate, beads (or other particles) in or on a substrate, projections from a substrate, ridges on a substrate or channels in a substrate. The sites of an array can be separate substrates each bearing at least one molecule. Different molecules attached to separate substrates can be identified according to the locations of the substrates on a surface to which the substrates are associated or according to the locations of the substrates in a liquid or gel. Such different molecules may have the same or different sequences. An array may include one or more wells, and a well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group, such as an amino acid side chain, C-terminal carboxylate, or N-terminal amine. The label may comprise at least one reactive group (e.g., a first reactive group and a second reactive group). The at least one reactive group may be configured to couple to a polypeptide. The at least one reactive group may be configured to couple to a support. The at least one reactive group may be coupled to or configured to couple to a detectable moiety. A label may provide a measurable signal.

As used herein, the term “polymer matrix” generally refers to a continuous phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid or gaseous species. For example, the term ‘polymer matrix’ may encompass desiccated hydrogels, hydrated hydrogels, and hydrogels containing glass fibers.

Biomolecule (e.g., peptide) sequence information may be obtained from a molecule (e.g., a polypeptide molecule) or from one or more portions of the molecule (e.g., polypeptide molecule). Biomolecule (e.g., peptide) sequencing may provide complete or sequence information (e.g., amino acid sequence information) for a biomolecule (e.g., peptide) sequence or a portion of a sequence. At least a portion of the sequence may be determined at the single molecule level. In some cases, partial sequence information, including for example, the relative positions of a specific type of biomolecule constituent (e.g. amino acid (e.g., lysine)) within a biomolecule or portion of a biomolecule, may be sufficient to uniquely identify an individual biomolecule. For example, a pattern of amino acids, such as, for example, X-X-X-Lys-X-X-X-X-Lys-X-Lys, which indicates the distribution of lysine molecules within an individual peptide molecule, may be searched against a proteome (e.g., database) of a given organism to identify the individual peptide molecule. Such information may be used to identify a macromolecule (e.g., protein) from which the biomolecule was derived, and may preclude the need to identify all portions of the biomolecule.

Biomolecule (e.g., peptide) sequencing may be used to acquire information (including, for example, amino acid sequence information) from individual biomolecules in a mixture of diverse biomolecules. In a non-limiting example, a plurality of peptides may be immobilized on a solid surface (including, for example, a glass slide, or a glass slide whose surface has been chemically modified, a plastic slide, a multi-well plate, a cassette), amino acids from the plurality of peptides may be coupled to fluorescent reporter moieties, and the fluorescent reporter moieties may be optically detected.

Numerous commercially available optical devices can be applied in this manner. For example, microscopes equipped with total internal reflection illumination and intensified charge-couple device (CCD) detectors may be adapted for sequencing methods disclosed herein. A high sensitivity CCD camera may be configured to simultaneously record the fluorescence intensity of multiple individual (e.g., single) peptide molecules distributed across a surface, and may be coupled to an image splitter to facilitate the simultaneous collection of multiple, distinct images (e.g., a first image comprising light of a first wavelength and a second image comprising light of a second wavelength). Using a motorized microscope stage with automated focus control to image multiple stage positions in the flow cell may allow thousands or more (e.g., millions) of individual single peptides (or more) to be sequenced in a single experiment. Sample processing for biomolecule sequencing (e.g., fluorosequencing) can comprise photochemistry, heterogeneous coupling and click chemistry, and solvent evaporation. In an aspect, the present disclosure provides methods and systems for sample processing. In some cases, the sample processing can be automated (e.g., transfer of a solid support to one or more reagents). In some cases, unlabeled peptide samples may be isolated and/or labeled for either immediate sequencing or storage prior to sequencing.

In an aspect, the present disclosure provides solutions to the aforementioned challenges by providing expeditious and facile methods for processing and analyzing a biomolecule (e.g., a polypeptide). Additionally, some aspects of the present disclosure provide compositions that facilitate effective biomolecule (e.g., peptide) characterization and analysis. Furthermore, in some aspects the present disclosure provides kits which enable effective biomolecule (e.g., polypeptide) analysis.

In another aspect, the present disclosure provides a method for processing a biomolecule. The method may comprise providing, at a first location, (i) a substrate and (ii) a substrate holder coupled to the substrate. The substrate may comprise the biomolecule coupled thereto. The substrate holder and the substrate may be automatically directed from the first location to a second location different from the first location. At the second location, the biomolecule may be processed to provide a processed biomolecule coupled to the substrate. The processing may comprise labelling the biomolecule to provide a labeled biomolecule.

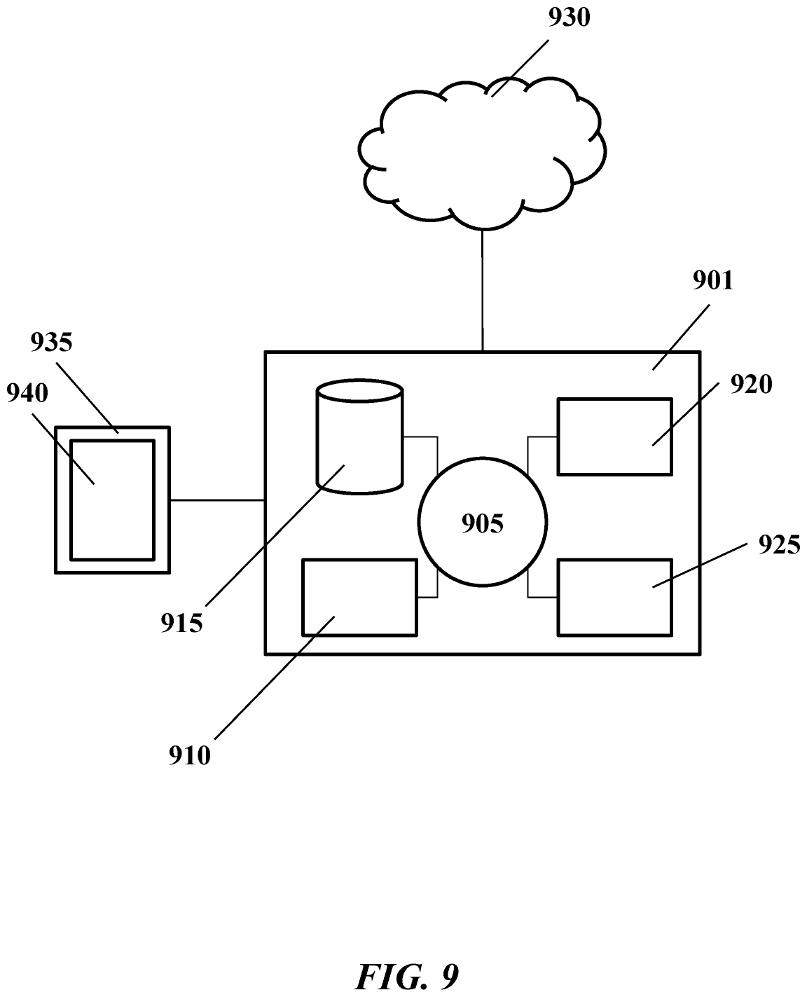

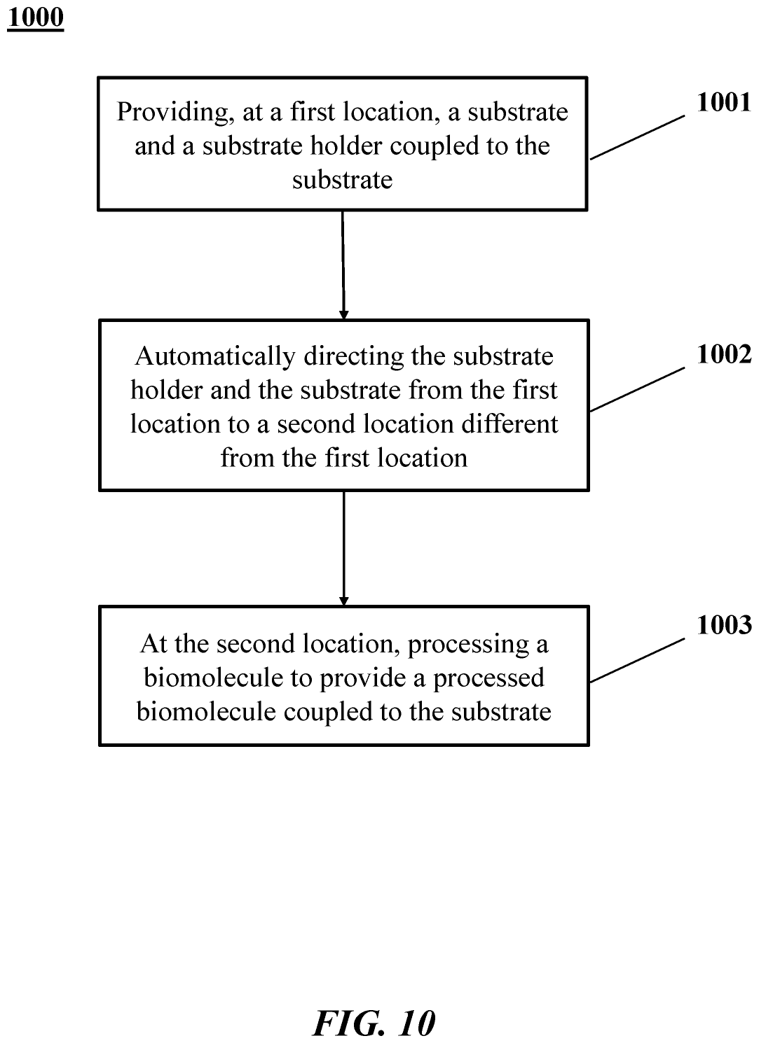

FIG. 10 schematically illustrates an example of a method 1000 for processing a biomolecule (“method”), according to an embodiment of the present disclosure. In an operation 1001, the method 1000 may comprise providing, at a first location, (i) a substrate and (ii) a substrate holder coupled to the substrate. The substrate may comprise the biomolecule coupled thereto. The method for processing may be completely automated (e.g., by a computer processor). For example, the method may be performed without human intervention. The automation may comprise performing and/or directing the operations of the method. The method may be partially automated (e.g., at least a portion of the method may be automated). For example, the directing the substrate and the processing the biomolecule may be automated while the providing the substrate may not be automated. The method for processing the biomolecule may not be automated. For example, a user can perform or direct the operation of the method. The automation may comprise use of one or more computer processors (e.g., a computer such as the computer system of FIG. 9 can direct the performance of the operations of the method).

The substrate may be coupled to the substrate holder at a first position of the substrate holder, the substrate holder may be coupled to an additional substrate comprising an additional biomolecule at a second position of the substrate holder, or a combination thereof. The coupling may comprise use of a support as described elsewhere herein. The substrate holder may comprise a lantern bar as described elsewhere herein. The substrate holder may be configured to couple to a plurality of substrates, which substrates are each coupled to different additional biomolecules. The substrate may be coupled to the substrate holder at a first position of the substrate holder, the substrate holder may be coupled to a plurality of additional substrates comprising a plurality of additional biomolecules at a plurality of positions of the substrate holder. The plurality of substrates may comprise at least about 1, 2, 3, 4, 5, 6, 7, 8, 9, 10, 11, 12, 13, 14, 15, 16, 17, 18, 19, 20, 25, 30, 32, 35, 40, 45, 48, 50, 60, 70, 80, 90, 96, 100, 150, 192, 200, 250, 300, 350, 384, 400, 500, 600, 700, 800, 900, 1,000, 1,250, 1,500, 1,536, or more substrates. The plurality of substrates may comprise at most about 1,536, 1,500, 1,250, 1,000, 900, 800, 700, 600, 500, 400, 384, 350, 300, 250, 200, 192, 150, 100, 96, 90, 80, 70, 60, 50, 48, 45, 40, 35, 32, 30, 25, 20, 19, 18, 17, 16, 15, 14, 13, 13, 11, 10, 9, 8, 7, 6, 5, 4, 3, 2, or fewer substrates.

The method 1000 may comprise coupling the biomolecule to the substrate. The coupling the biomolecule to the substrate may be performed prior to operation 1001. The coupling may occur at an additional location different from the first location or the second location. For example, the coupling can be performed outside of the automated robotic handling system. In this example, a user can perform the coupling to the substrate. In some cases, the coupling occurs at the first location (e.g., a first well). For example, the biomolecule can be added into a well of a well plate, and the substrate can be introduced to the well to bind the biomolecule. The biomolecule may comprise a protein, a peptide, a polypeptide, a lipid, a carbohydrate, a metabolite, a nucleic acid molecule, an antibody, an antibody fragment, an antigen, or the like, or any combination thereof. The biomolecule may be obtained from a biological sample. For example, the sample may comprise the biomolecule as well as additional molecules. Nonlimiting examples of a samples include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. A sample may be a cell-free sample. Such cell-free sample may include DNA and/or RNA.

The substrate may comprise a bead. The bead may be a polymer bead (e.g., a polystyrene bead, a latex bead, etc.), a metal bead (e.g., a metal nanoparticle), a semiconductor bead (e.g., a quantum dot), a biological bead (e.g., at least one protein), a glass bead (e.g., a silicon dioxide bead), or the like, or any combination thereof. The bead may be associated with a substrate. For example, the bead can be bound to a surface of a substrate. In another example, the bead can be placed on a substrate. The bead may have a diameter of at least about 1, 5, 10, 25, 50, 75, 100, 150, 200, 250, 300, 400, 500, 750, 1,000, or more micrometers. The bead may have a diameter of at most about 1,000, 750, 500, 400, 300, 250, 200, 150, 100, 75, 50, 25, 10, 5, 1, or less micrometers. The substrate may comprise a lantern. The lantern may comprise one or more resins (e.g., be formed of one or more resins). The lantern may comprise one or more surface linkers as described elsewhere herein. For example, the lantern can be configured with a linker linking the lantern to a biomolecule. The lantern may comprise a plurality of surfaces held together to increase the surface are available on the lantern. For example, a plurality of rings can be placed in a stacked configuration with small gaps between the rings, and the rings can be linked together in the stack. The lantern may be a lantern as described in Example 2. The lantern may comprise polymers (e.g., plastics, polyethylene, polytetrafluoroethylene, etc.), metals (e.g., iron, steel, aluminum, zinc, etc.), alloys, or the like, or any combination thereof. The lantern may be configured with one or more capture reagents as described elsewhere herein. For example, the lantern can be configured to capture a protein. The lantern may be a substrate. The substrate may comprise a solid support. The solid support may, for example a slide, a bead, a well, a pore, or the like, or any combination thereof. The substrate may comprise a polyethylene glycol (PEG) linker, polyacrylate, polyamide, polystyrene, polyethylene, tetrafluoroethylene, or the like, or any combination thereof.

The biomolecule may comprise a peptide, and the labelling may comprise coupling a label to an amino acid of the peptide. The labelling may comprise coupling a plurality of labels to a plurality of amino acids of the peptide. For example, a plurality of different labels can each be attached to a plurality of different types of amino acids of the peptide. The labels may be as described elsewhere herein. The labeling may comprise a first reactive group that is configured to couple to a second reactive group that is coupled to a reporter moiety configured to emit a signal, a protecting group configured to prevent coupling between the label and the second reactive group, or a combination thereof. The first reactive group may be configured as a bridge between the amino acid and a reporter moiety. For example, the first reactive group can be configured to functionalize the amino acid to improve a binding efficacy or signal efficiency of the reporter moiety. In this example, if the reporter moiety were to be tethered directly to the amino acid, the signal received may be lower than in the case where the first reactive group is used. The reporter moiety may be as described elsewhere herein. The reporter moiety may comprise alight-based reporter moiety (e.g., emission intensity, emission wavelength, emission photoluminescent lifetime, absorption intensity, absorption wavelength, absorptive state lifetime, a change thereof, etc.), a magnetic reporter moiety (e.g., a presence or absence of a magnetic field, etc.), an electrical reporter moiety (e.g., effecting the current, voltage, resistance, capacitance, or change thereof, etc.), or the like, or any combination thereof. The protecting group may be configured to prevent the coupling by binding to the label or the second reactive group, thereby blocking the coupling by providing a non-reactive functionalization. For example, for a coupling based on ionic attraction, the protecting group can ionically bind to the label or the second reactive group to prevent binding.

The substrate may comprise a peptide capture reagent. Examples of peptide capture reagents include, but are not limited to a substituted heterocycle (e.g., pyridinecarbaldehyde (PCA), etc.), a biotin, a streptavidin, a strep-tag II, a linker, a functional group that can react with a molecule (e.g., an aldehyde, a phosphate, a silicate, an ester, an acid, an amide, an alkyne, an azide, an aldehyde dithiolane, etc.), or the like, or any combination thereof. The peptide capture reagent may comprise a cleavable linker. For example, the peptide capture reagent can be configured to be releasable upon cleaving of a linker. The cleavable linker may comprise one or more disulfide linkers, photocleavable linkers (e.g., linkers that can cleave upon light illumination), enzymatically cleavable linkers (e.g., linkers configured to be cleaved by one or more enzymes), chemically cleavable linkers (e.g., linkers cleavable under acidic conditions, etc.), or the like, or any combination thereof. The peptide capture reactant may comprise an N-terminal capture reagent as described elsewhere herein.

In an operation 1002, the method 1000 may comprise automatically directing the substrate holder and the substrate from the first location to a second location different from the first location. The first and second locations may be locations within an automated robotic handling system as described elsewhere herein.

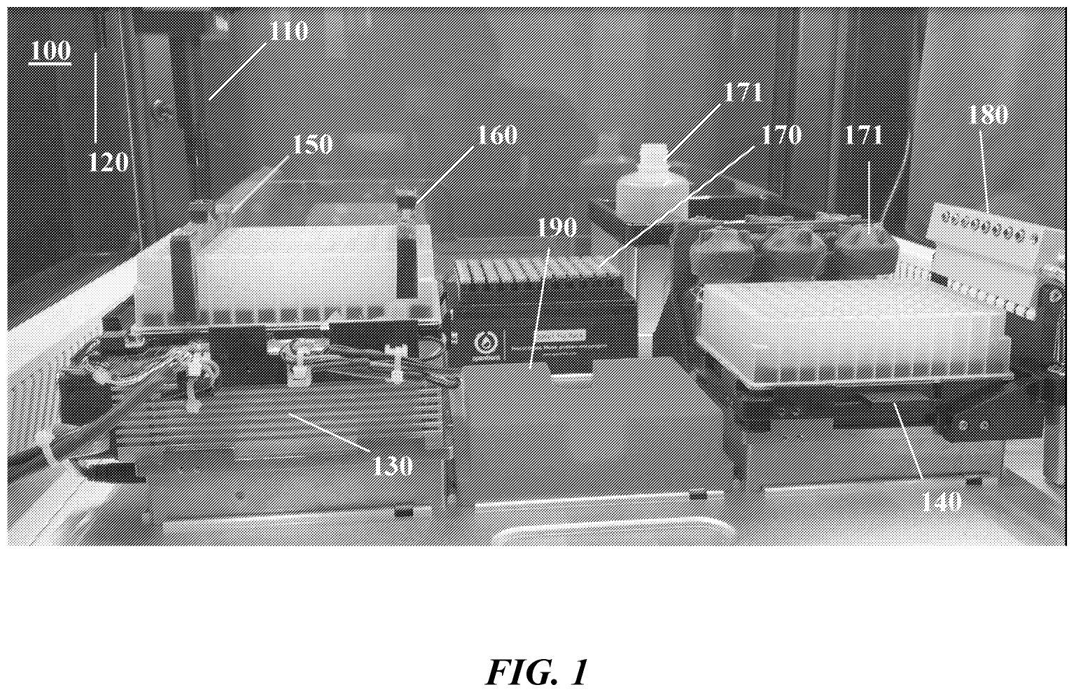

Operation 1002 may be performed using a robotic system (e.g., the automated robotic handling system of FIG. 1) configured to direct the substrate holder from the first location (e.g., a first reagent container) to the second location (e.g., a second reagent container). For example, an arm can be configured to move the substrate via the substrate holder from a first well to a second well. In some cases, the robotic system comprises a fluid handling unit (e.g., a pipette attached to an arm), a movable stage (e.g., an arm, a shaker element), an evaporation unit, a vacuum (e.g., a vacuum associated with a vacuum bar), a gas pump (e.g., a gas pump bar), a light source, a temperature control unit, a shaker, a fan, or the like, or any combination thereof as described elsewhere herein. The temperature control unit may comprise a compressive temperature control unit (e.g., a compressor-based refrigerator), a thermoelectric temperature control unit, a fluid bath temperature control unit (e.g., a temperature-controlled water bath), a resistive electrical heater control unit, or the like, or any combination thereof. The light source may be as described elsewhere herein.

The robotic system may comprise a fluid handling unit. The method may comprise, prior to operation 1002, providing a reagent container (e.g., a well). The well may be a part of a multi-container holder (e.g., multi well plate) as described elsewhere herein. In some cases, the fluid handling unit may be used to provide reagents to the well. The well and/or the reagent may be as described elsewhere herein. For example, the well can be contained within a well plate. In another example, the reagents may comprise labelling agents. The labelling agents may comprise labeling agents as described elsewhere herein (e.g., fluorescent labelling agents, magnetic labeling agents, electrical labeling agents, etc.). The labelling agents may be configured to label one or more components of the biomolecule. For example, for a peptide biomolecule, the labelling agents can be configured to label one or more amino acids. In another example, for a nucleic acid biomolecule, the labelling agents can be configured to label one or more nucleotides. The labelling agents may comprise a labelling agent configured to label a lysine residue, a cysteine residue, a glutamic acid residue, an aspartic acid residue, a tyrosine residue, an arginine residue, a histidine residue, a threonine residue, a serine residue, a proline residue, a glutamine residue, or a tryptophan residue. The labelling agents may comprise a plurality of labelling agents where each labelling agent of the plurality of labelling reagents is configured to label a different residue. For example, a first labelling agent with a first fluorescence emission wavelength can label a lysine residue while a second labelling agent with a second fluorescence emission wavelength can label a serine residue. The labeling agents may be configured to label one or more post-translational modifications such as, for example, phosphorylation, glycosylation, ubiquitination, methylation, or the like, or any combination thereof. For example, the label can be configured to label peptides that have been post-translationally modified and not peptides that have not been post-translationally modified.

The method may comprise using a movable stage to move the reagent container (e.g., well) relative to the fluid handling unit. For example, the well can be a part of a well plate, and the well plate can be moved using an arm of an automated robotic handling system. The robotic system may comprise a feedback mechanism configured to regulate the fluid handling unit, the movable stage, the evaporation unit, the vacuum, the gas pump, the light source, the temperature control unit, the fan, or any combination thereof. The feedback mechanism may comprise one or more sensors (e.g., temperature sensors, position sensors, pressure sensors, light sensors, chemical sensors, etc.). The feedback mechanism may be coupled to a computer system. The computer system may be configured to perform the automated portions of method 1000 using data from the feedback mechanism to ensure proper execution of the method.

In an operation 1003, the method 1000 may comprise, at the second location, processing the biomolecule to provide a processed biomolecule coupled to the substrate. The processing may comprise labelling the biomolecule to provide a labelled biomolecule. The labelling may comprise use of a labelling agent as described elsewhere herein. In some cases, the processing can comprise processing the biomolecule while the biomolecule is affixed to a substrate. In some cases, the processing can comprise processing the biomolecule while the biomolecule is not affixed to a substrate. In some cases, the processing can comprise processing the biomolecule while the biomolecule is affixed to the substrate and not affixed to the substrate in different periods of the processing.

The processing may comprise contacting the substrate coupled to the biomolecule with reagents in the reagent container (e.g., well) to provide the labelled biomolecule. For example, the substrate can be moved via an arm into a well comprising labelling reagents and processed to affix the labels to the biomolecule. Operation 1003 may comprise using the shaker to mix the reagents in the well. For example, the reagents and the substrate can be placed in a well plate positioned on the shaker, and the shaker can be activated to shake the substrate and the reagents to perform the processing. The light source may be used to attach one or more reagents of the reagents to the biomolecule, thereby providing the labelled biomolecule. For example, the light source can be used to perform a radical initiation that activates a linker configured to link an acceptor molecule to the biomolecule.

The robotic system can comprise the evaporation unit, and the processing of operation 1003 may comprise use of a solvent. The method may comprise, subsequent to operation 1003, using the evaporation unit to evaporate the solvent from the labelled biomolecule. The evaporation unit may be an evaporation bar as described elsewhere herein. For example, subsequent to a labelling reaction, the evaporation unit can be positioned adjacent to a well comprising the processed biomolecule and activated to remove solvent from the well to provide a dried biomolecule. In this example, solvent can be added to the dried biomolecule, thus resuspending the processed biomolecule, and a lantern substrate can be introduced to the well to bind to the processed biomolecule. In another example, a biomolecule can be bound to a lantern substrate and processed while bound to the substrate. In this example, the evaporation unit can be positioned over the substrate to remove a solvent from the substrate. The evaporation unit may comprise the vacuum. The evaporation unit may be attached to an external vacuum. For example, the evaporation unit can be connected to a vacuum pump external to the robotic system. The evaporation unit may comprise the gas pump. For example, the evaporation unit can connect to a gas source configured to provide gas to a well. In this example, the gas can be blown over the well to evaporate a solvent from the well. The gas pump may be coupled to an inert gas stream (e.g., nitrogen, argon, neon, helium, etc.), a reactant gas stream (e.g., a gas configured to perform at least a portion of a chemical reaction in the well), or the like, or any combination thereof. Operation 1003 may be performed at a controlled temperature. The controlled temperature may be at least about 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, or more degrees Celsius. The controlled temperature may be at most about 75, 70, 65, 60, 55, 50, 45, 40, 39, 38, 37, 36, 35, 34, 33, 32, 31, 30, 29, 28, 27, 26, 25, 24, 23, 22, 21, 20, 15, 10, 5, 0, or less degrees Celsius.

A light source may be provided at the second location. The light source may be as described elsewhere herein. For example, the light source can be a light emitting diode (LED) light source. In another example, the light source can be a laser light source. The light source can be configured to illuminate a predetermined pattern. The light source may be configured to couple into a well plate (e.g., a multi-well plate) and illuminate one or more wells of the well plate. For example, the light source can be configured to illuminate some wells of a well plate while not illuminating other wells of the well plate. The predetermined pattern may be a time resolved pattern. For example, a first set of wells can be illuminated at a first time, while a second set of wells may be illuminated at a second time. The light source may be used to conduct a photoreaction to provide the labelled biomolecule. For example, the light source can initiate a radical photoreaction. The photoreaction may comprise decarboxylative alkylation, click chemistry, photoisomerization, radical polymerization, radical addition, or the like, or any combination thereof.

The method may comprise removing the labelled biomolecule from the substrate. The removal may be removal of the biomolecule from the substrate into a solution. For example, the labelled biomolecule can be hydrolyzed from the substrate into solution. The removing may comprise contacting the substrate with an acid or an acid mixture (e.g., a mixture of an acid with a solvent as described elsewhere herein). Examples of acids include, but are not limited to, organic acids (e.g., trifluoroacetic acid (TFA), trifluoromethanesulfonic acid, acetic acid, formic acid, etc.), mineral acids (e.g., hydrochloric acid, hydrobromic acid, etc.), oxidizing acids (e.g., nitric acid, sulfuric acid, etc.), or the like. The evaporation bar may be used to remove the acid from the labelled biomolecule. For example, the evaporation bar can provide a reduced pressure atmosphere over a well to remove the acid. In another example, the evaporation bar can provide a flow of a gas over the well to enhance evaporation from the well. The removing may comprise contacting the substrate with a base. Examples of bases include, but are not limited to, organic bases (e.g., anions or salts of organic acids, sodium carbonate, etc.), mineral bases (e.g., sodium hydroxide, potassium hydroxide, etc.), or the like. The method may comprise storing the labelled biomolecule. For example, after the processing in completed, the biomolecule can be stored in an output stage as described elsewhere herein. The labelled biomolecule can be stored for later processing and/or sequencing. The method may comprise evaporating the removed biomolecule. For example, the removed biomolecule can be suspended in a solvent, and the solvent can be removed from the biomolecule. The evaporating may be performed using an evaporation unit as described elsewhere herein. For example, the evaporation unit can be coupled to a gas source or a vacuum. In some cases, the gas source can be an inert gas stream (e.g., a nitrogen stream, a noble gas stream, etc.). In some cases, the evaporation can be performed with a heated gas (e.g., a heated gas stream). The use of a heated gas stream may improve evaporation speeds as compared to a non-heated gas stream.

The method may further comprise subjecting the labelled biomolecule to sequencing. The sequencing may be as described elsewhere herein. For example, the sequencing can comprise fluorosequencing a protein. The labelled biomolecule may be a labelled peptide or protein and the sequencing may comprise protein sequencing. The protein sequencing may comprise fluorosequencing. The method may comprise, prior to the sequencing, coupling the labelled biomolecule to a flow cell. The flow cell may comprise a linker as described elsewhere herein configured to couple the labelled biomolecule to the flow cell. For example, the labelled biomolecule can be removed from a substrate and subsequently contacted to a flow cell configured with a binder configured to bind the labelled biomolecule to the flow cell.

In another aspect, the present disclosure provides a system for processing a biomolecule. The system may comprise a substrate comprising the biomolecule coupled thereto. The system may comprise a substrate holder configured to couple to the substrate. The system may comprise an automated robotic handling system configured to automatically direct the substrate holder from a first location to a second location. The system may comprise reagents for labelling the biomolecule. The system may comprise an automated robotic handling system as described in FIG. 1.

In some cases, the substrate is a bead as described elsewhere herein. In some cases, the substrate is a lantern as described elsewhere herein. The substrate may be coupled to the substrate holder at a first position of the substrate holder. The substrate holder may be coupled to an additional substrate comprising an additional biomolecule at a second position of the substrate holder. For example, the substrate holder can be a lantern bar as described elsewhere herein, and the lantern bar can comprise a plurality of substrates. The substrate may be coupled to the substrate holder at a first position of the substrate holder. The substrate holder may be coupled to a plurality of additional substrates comprising a plurality of additional biomolecules at a plurality of positions of the substrate holder as described elsewhere herein. The plurality of substrates may be as described elsewhere herein. For example, the plurality of substrates may comprise at least about 1, 2, 3, 4, 5, 6, 7, 8, 9, 10, 11, 12, 13, 14, 15, 16, 17, 18, 19, 20, 25, 30, 32, 35, 40, 45, 48, 50, 60, 70, 80, 90, 96, 100, 150, 192, 200, 250, 300, 350, 384, 400, 500, 600, 700, 800, 900, 1,000, 1,250, 1,500, 1,536, or more substrates.

The system may comprise a light source at the second location. The light source may be as described elsewhere herein. For example, the light source can be a light emitting diode (LED) light source. In another example, the light source can be a laser light source. The light source can be configured to illuminate a predetermined pattern. The light source may be configured to couple into a well plate (e.g., a multi-well plate) and illuminate one or more wells of the well plate. For example, the light source can be configured to illuminate some wells of a well plate while not illuminating other wells of the well plate. The predetermined pattern may be a time resolved pattern. For example, a first set of wells can be illuminated at a first time, while a second set of wells may be illuminated at a second time.

The system may comprise a robotic system (e.g., an automated robotic handling system) configured to direct the substrate holder from a first location to a second location as described elsewhere herein. In some cases, the robotic system comprises a fluid handling unit (e.g., a pipette attached to an arm), a movable stage (e.g., an arm, a shaker element), an evaporation unit, a vacuum (e.g., a vacuum associated with a vacuum bar), a gas pump (e.g., a gas pump bar), a light source, a temperature control unit, a shaker, a fan, or the like, or any combination thereof as described elsewhere herein. The robotic system may comprise a feedback mechanism configured to regulate the fluid handling unit, the movable stage, the evaporation unit, the vacuum, the gas pump, the light source, the temperature control unit, the fan, or any combination thereof. The feedback mechanism may comprise one or more sensors (e.g., temperature sensors, position sensors, pressure sensors, light sensors, chemical sensors, etc.). The feedback mechanism may be coupled to a computer system. The computer system may be configured to perform the automated portions of method 1000 using data from the feedback mechanism to ensure proper execution of the method. The feedback mechanism may comprise an open loop feedback system. For example, the feedback mechanism can be configured to operate the robotic system based on a given set of controls without using input data to adjust the controls. The feedback mechanism may comprise a closed loop feedback system. For example, sensor data can be used by the feedback mechanism to adjust the parameters of operation for the robotic system.

The robotic system may comprise an evaporation unit (e.g., a gas pump bar, a vacuum bar, or a combination thereof) as described elsewhere herein. For example, the evaporation unit can comprise a vacuum, a gas pump, or a combination thereof. In this example, the gas pump can be coupled to an inert gas stream. The robotic system may be configured to accommodate a multi-well plate. The multi-well plate may comprise at least about 1, 2, 3, 4, 5, 6, 7, 8, 9, 10, 11, 12, 13, 14, 15, 16, 17, 18, 19, 20, 25, 30, 32, 35, 40, 45, 48, 50, 60, 70, 80, 90, 96, 100, 150, 192, 200, 250, 300, 350, 384, 400, 500, 600, 700, 800, 900, 1,000, 1,250, 1,500, 1,536, or more wells. The multi-well plate may comprise at most about 1,536, 1,500, 1,250, 1,000, 900, 800, 700, 600, 500, 400, 384, 350, 300, 250, 200, 192, 150, 100, 96, 90, 80, 70, 60, 50, 48, 45, 40, 35, 32, 30, 25, 20, 19, 18, 17, 16, 15, 14, 13, 13, 11, 10, 9, 8, 7, 6, 5, 4, 3, 2, or fewer wells.

The reagents for labelling may comprise labelling agents configured to label one or more amino acids of a peptide as described elsewhere herein. For example, the labelling agents can comprise a labelling agent configured to label a lysine residue, cysteine residue, glutamic acid residue, aspartic acid residue, tyrosine residue, arginine residue, histidine residue, threonine residue, serine residue, proline residue, glutamine residue, or tryptophan residue. The labelling agents may be configured to label post-translational modifications (e.g., phosphorylation, glycosylation, ubiquitination, methylation, etc.). The labelling agents may comprise agents for performing decarboxylative alkylation, click chemistry, or the like, or any combination thereof. The labelling agents may comprise a first reactive group that may be configured to couple to a second reactive group that may be coupled to a reporter moiety configured to emit a signal as described elsewhere herein. The labelling agents may comprise a protective group configured to prevent coupling between the label and the second reactive group as described elsewhere herein.

The biomolecule may be as described elsewhere herein. For example, the biomolecule can comprise a peptide. The reagents may comprise reagents for removing the labelled biomolecule from the substrate as described elsewhere herein. For example, the reagents can comprise an acidic buffer with a pH of 1 to 4, such as formic acid, trifluoroacetic acid (TFA), or glycine hydrochloride. The system may comprise one or more storage units for storing the labelled biomolecule as described elsewhere herein. For example, the labelled biomolecule can be stored in a well. In another example, the labelled biomolecule can be stored in a vial. The system may comprise a flow cell configured to couple to the labelled biomolecule as described elsewhere herein. The flow cell may be housed within the system. For example, the flow cell can be contained within an automated robotic handling system. In this example, the labelled biomolecule can be transferred from a well in the handling system into the flow cell, and the flow cell can be taken and inserted into a fluorosequencing machine. The labelled biomolecule may comprise a labelled peptide or protein. The flow cell may comprise a linker configured to couple to the labelled biomolecule as described elsewhere herein. The substrate may comprise a peptide capture reagent as described elsewhere herein. For example, the peptide capture reagent may comprise a cleavable linker. In another example, the peptide capture reagent can comprise an N-terminal capture reagent. In this example, the N-terminal capture reagent can be pyridinecarbaldehyde (PCA) or a derivative thereof. The substrate may comprise a poly-ethylene glycol (PEG) linker, polyacrylate, polyamide, or polystyrene as described elsewhere herein. The biomolecule may be as described elsewhere herein. For example, the biomolecule can be obtained from a biological sample (e.g., a cell, a tissue sample, a blood sample, etc.).

In another aspect, the present disclosure provides a system for processing a biomolecule. The system may comprise a substrate holder configured to couple a substrate comprising the biomolecule coupled thereto. The system may comprise a robotic arm coupled to the substrate holder. The system may comprise at least one computer processor configured to perform executable instructions and a memory comprising the executable instructions which, when executed by the at least one computer processor, causes the at least one computer processor to implement a method comprising automatically instructing the robotic arm to direct the substrate holder and the substrate from a first location to a second location different from the first location. At the second location, the biomolecule may be labelled to provide a labelled biomolecule coupled to the substrate. The computer processor may be as described elsewhere herein, such as, for example, FIG. 9.

The system may comprise a fluid handling unit as described elsewhere herein. The method may comprise instructing the fluid handling unit to provide reagents in a vessel in the second location. For example, the fluid handing unit can be directed to uptake reagents from a bottle into a pipette tip, move the pipette tip to the second location, and provide the reagents to the second location. The reagents may comprise labelling agents as described elsewhere herein. The system may comprise a movable stage as described elsewhere herein. The method may further comprise instructing the movable stage to move from the first location to the second location, from the second location to a third location different from the second location, or a combination thereof. The method may comprise at least about 1, 5, 10, 25, 50, 75, 100, or more movement operations. The method may comprise at most about 100, 75, 50, 25, 10, 5, or fewer movement operations. The method may comprise movement between at least about 1, 2, 3, 4, 5, 6, 7, 8, 9, 10, 11, 12, 13, 14, 15, or more locations within the automated handling system. The method may comprise movement between at most about 15, 14, 13, 12, 11, 10, 9, 8, 7, 6, 5, 4, 3, 2, or fewer locations within the automated handling system.

The system may comprise an evaporation unit as described elsewhere herein. For example, the evaporation unit can be coupled to a vacuum or gas pump. The method may comprise instructing the robotic arm to move the evaporation unit. In some cases, the evaporation unit comprises a positioning arm, and the method can comprise instructing the positioning arm to move the evaporation unit. The system may comprise a light source as described elsewhere herein. The method may comprise electronically controlling the light source. For example, the method may comprise turning on the light source, turning off the light source, increasing an intensity of the light source, decreasing an intensity of the light source, changing a wavelength of the light source, or the like, or any combination thereof. The system may comprise a temperature control unit as described elsewhere herein. For example, the temperature control unit may comprise a fan or a thermoelectric component. The method may comprise controlling the temperature control unit to obtain a temperature within a range of temperatures. The range of temperatures may be a temperature range as defined by any two of the following values: −50, −45, −40, −35, −30, −25, −20, −15, −10, −5, 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 degrees Celsius.

FIG. 1 illustrates an automated robotic handling system 100. The automated robotic handling system 100 can comprise one or more arms 110 and 120. The automated robotic handling system may comprise at least about 1, 2, 3, 4, 5, 6, 7, 8, 9, 10, or more arms. The automated robotic handling systems may comprise at most about 10, 9, 8, 7, 6, 5, 4, 3, 2, or fewer arms. The arms may be configured to move one or more other elements of the automated robotic handling system (e.g., moving a lantern bar from one area to another, moving a plate within the handling system, etc.), dispensing one or more reagents (e.g., pipetting one or more liquid reagents, dispensing one or more solid reagents, etc.), moving one or more samples (e.g., moving a sample into a plate, removing a processed sample from the plate, etc.), or the like, or any combination thereof. A single arm may be configured to perform a single task (e.g., one arm is configured to dispense liquid reagents while a second arm is configured to move a lantern bar). A single bar may be configured to perform a plurality of tasks (e.g., be configured to both move a reagent cap from a bottle as well as dispense the reagents from the bottle).

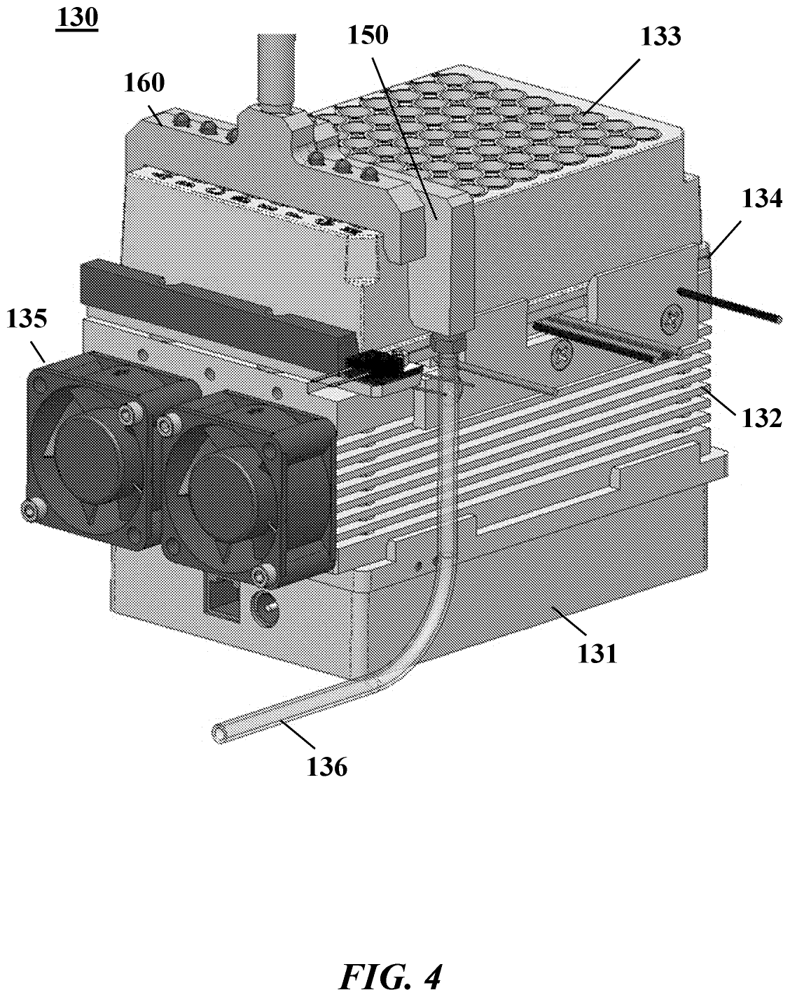

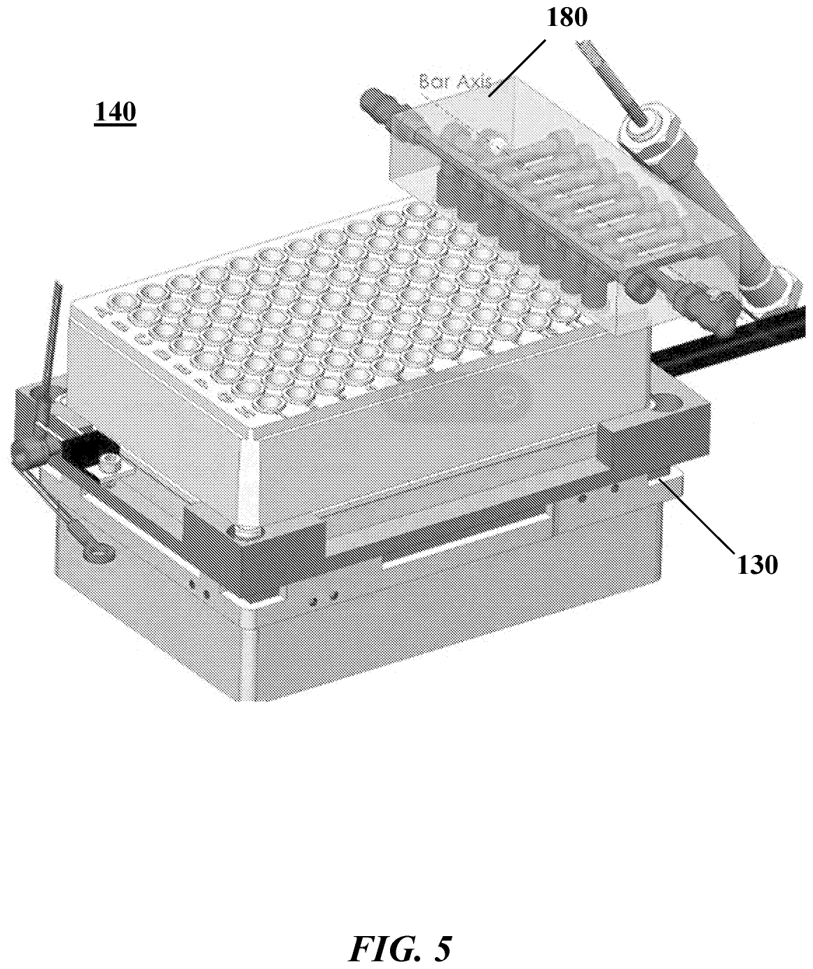

The automated robotic handling system 100 may comprise one or more reaction stages 130 and 140. The one or more reaction stages may be one or more movable stages. For example, the stages can be moved within the handling system. The handling system may comprise at least about 1, 2, 3, 4, 5, 6, 7, 8, 9, 10, or more reaction stages. The automated robotic handling systems may comprise at most about 10, 9, 8, 7, 6, 5, 4, 3, 2, or fewer reaction stages. For example, the system 100 of FIG. 1 can comprise two reaction stages. The reaction stages may be as described elsewhere herein, such as, for example, with regards to FIGS. 4-5.

The automated robotic handling system 100 may comprise at least one gas pump bar 150. The gas pump bar may be configured to flow a gas over one or more wells of a plate positioned on a reaction stage. For example, the gas pump bar can be connected to a pressurized gas line and provide the gas to a plurality of wells via a plurality of outlets. The gas pump bar con be configured to provide a gas as described elsewhere herein to wells of a plate (e.g., inert gas, reagent gas, etc.). The gas may be provided by an arm (e.g., arm 110). For example, the arm can be connected to the gas source, and can provide the gas through the top of the gas pump bar. The gas may be provided through a separate connection (e.g., through a line on a side of the gas pump bar). The gas pump bar may be movable throughout the automated robotic handling system. For example, the gas pump bar can be placed on either reaction stage 130 or reaction stage 140 at different times during a sample processing process. The gas pump bar may be made of one or more of polymers (e.g., plastics, polyethylene, polytetrafluoroethylene, etc.), metals (e.g., iron, steel, aluminum, zinc, etc.), alloys, or the like, or any combination thereof.

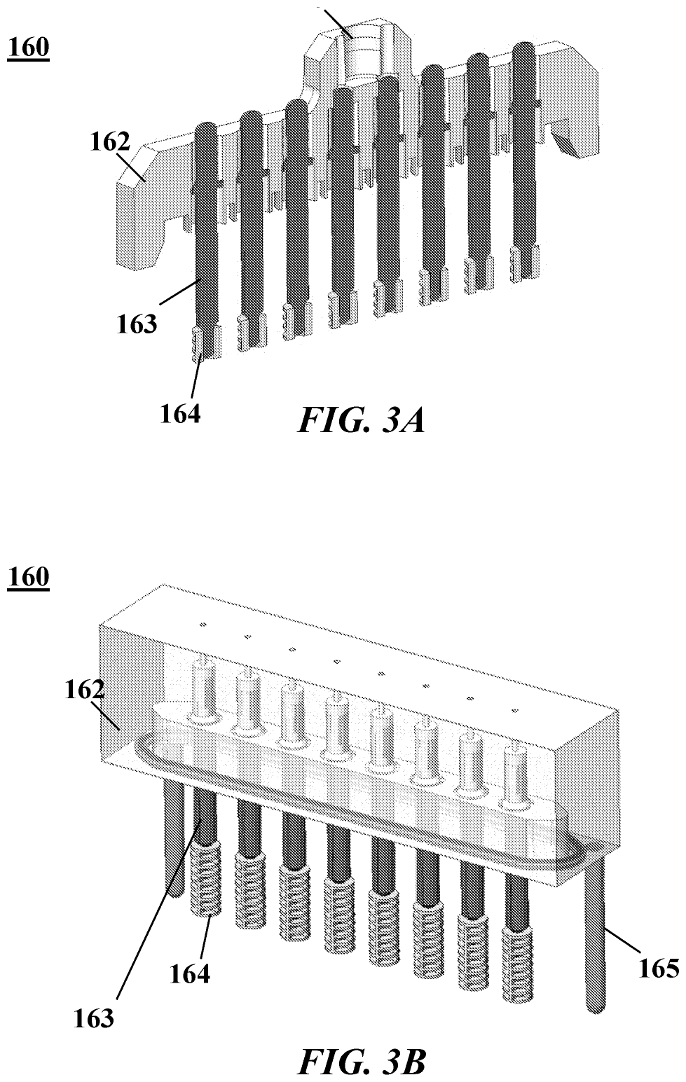

The automated robotic handling system 100 may comprise one or more lantern bars 160. The one or more lantern bars may be as described elsewhere herein (e.g., a lantern bar of FIGS. 3A-3B). The automated robotic handling system may comprise at least about 1, 2, 3, 4, 5, 6, 7, 8, 9, 10, or more lantern bars. The automated robotic handling systems may comprise at most about 10, 9, 8, 7, 6, 5, 4, 3, 2, or fewer lantern bars. The handling system may comprise one or more lantern bars in use during sample processing and one or more lantern bars held in reserve for future processing. For example, an array of lantern bars can be stored in the automated robotic handling system. The lantern bar can be transported through the automated robotic handling system via one or more of the arms (e.g., arm 110 or 120).

The automated robotic handling system 100 may comprise one or more reagents and/or consumables 170. The one or more reagents may comprise one or more reagents for use in a fluorosequencing sample preparation process. Examples of reagents include, but are not limited to, labeling reagents (e.g., labeling moieties), rinse reagents (e.g., water, alcohols, ethers, ketones, aldehydes, non-polar organic reagents, or the like, or any combination thereof), digestion reagents (e.g., GluC in digestion buffer), extraction reagents (e.g., reagents configured to extract target molecules from a sample, surfactants, etc.), substrates (e.g., functionalized glass slides (e.g., functionalized glass slides configured for use in fluorosequencing applications)), or the like, or any combination thereof. Examples of consumables include, but are not limited to, pipette tips, lantern bars, cleaning swabs, well caps, weighing boats or paper, test strips, or the like, or any combination thereof. The reagents may be a part of a reagent kit. For example, all of the reagents for a particular processing operation can be provided to the handing system as a single kit.

The kit may comprise a protein capture agent. The protein capture agent may be configured to couple to a terminus (e.g., N-terminus) of the peptide. The protein capture agent may comprise a solid support coupled to a cleavable linker. The protein capture agent may be coupled to a solid support by a cleavable linker. The solid support may comprise a bead, an array, a slide, a polymer matrix, or any combination thereof. The cleavable linker may be cleavable by an enzyme. The cleavable linker may be a chemically cleavable linker. The cleavable linker may be a photocleavable linker. The cleavable linker may be capable of being cleaved by a change in pH. The cleavable linker may comprise an aldehyde. The aldehyde may be pyridinecarbaldehyde (PCA) or a derivative of PCA.

A capture reagent may react with at least one peptide or protein. A capture reagent may react with the N-terminus of at least one peptide or protein. A capture reagent may react with the C-terminus of at least one peptide or protein. A capture reagent may react with one peptide or protein. A capture reagent may react with the N-terminus of one peptide or protein. A capture reagent may react with the C-terminus of one peptide or protein. Each peptide or protein of a cell may be captured by a plurality of capture reagents. The support may further comprise a capture reagent that can capture a molecule that is not a peptide or protein. The support may further comprise a capture reagent that can capture a nucleic acid molecule. The support may further comprise a capture reagent that can capture a ribonucleic acid molecule.

The reporter (or reporter moiety) may be configured to emit a signal. The reporter (or reporter moiety) may comprise a dye. The dye may be selected from the group consisting of fluorescent dyes, phosphorescent dyes, chemiluminescent dyes, pigments, and photoswitchable reporters. The reporter (or reporter moiety) may comprise a fluorescent dye. The reporter may be configured to emit the signal upon excitation. The reporter may be a fluorescent protein molecule.

The kit may comprise a surface attachment agent. The surface attachment agent may comprise an alkyne or an azide. The surface attachment agent may be configured to couple to a C-terminus of a peptide. A kit may comprise a support to which the surface attachment agent attaches. In some cases, the support is a slide. The slide may be a glass slide. The slide may be a microscopic slide.

The kit may comprise additional agents useful for carrying out a reaction, handling a peptide or any of the reagents described herein, or performing analysis. A kit may comprise one or more species from the group consisting of proteases, digestion reagents, solid support beads, or any combination thereof. A kit may also comprise small molecules, buffers, and solvents useful for carrying out a reaction. A kit may come pre-packaged in a container set. The prepackaging may be a cassette configured to be used in any sequencing platform. The kit may comprise a substrate comprising a plurality of volumes (e.g., a well plate comprising a plurality of wells). The kit may comprise one or more calibration proteins (e.g., proteins with a known sequence and/or concentration for use as a standard). The kit may comprise instructions for carrying out the methods described herein (e.g., sample processing methods, fluorosequencing methods, etc.).

A labeling moiety used in the instant application may be configured to withstand conditions for removing one or more of the amino acid residues. Some non-limiting examples of potential labeling moieties that may be used in the instant methods include, for example, those which emit a fluorescence signal in the red to infrared spectra such as an Alexa Fluor dye, an Atto dye, Janelia Fluor dye, a rhodamine dye, or other similar dyes. Examples of each of these dyes which were capable of withstanding the conditions of removing the amino acid residues include Alexa Fluor 405, Rhodamine B, tetramethyl rhodamine, Janelia Fluor 549, Alexa Fluor 555, Atto647N, and (5)6-naphthofluorescein. The labeling moiety may be a fluorescent peptide or protein or a quantum dot.

The reagents may be stored in one or more bottles 171 as shown in FIG. 1. The one or more bottles may comprise one or more caps. The caps may be movable by use of an arm of the handling system (e.g., arm 110 or 120). A first arm may be configured to remove the cap from the bottle while a second arm is configured to remove the reagent from the bottle (e.g., by pipette). Waste may be supplied to a waste container within the handling system or supplied to a waste disposal area that removes the waste from the handling system.

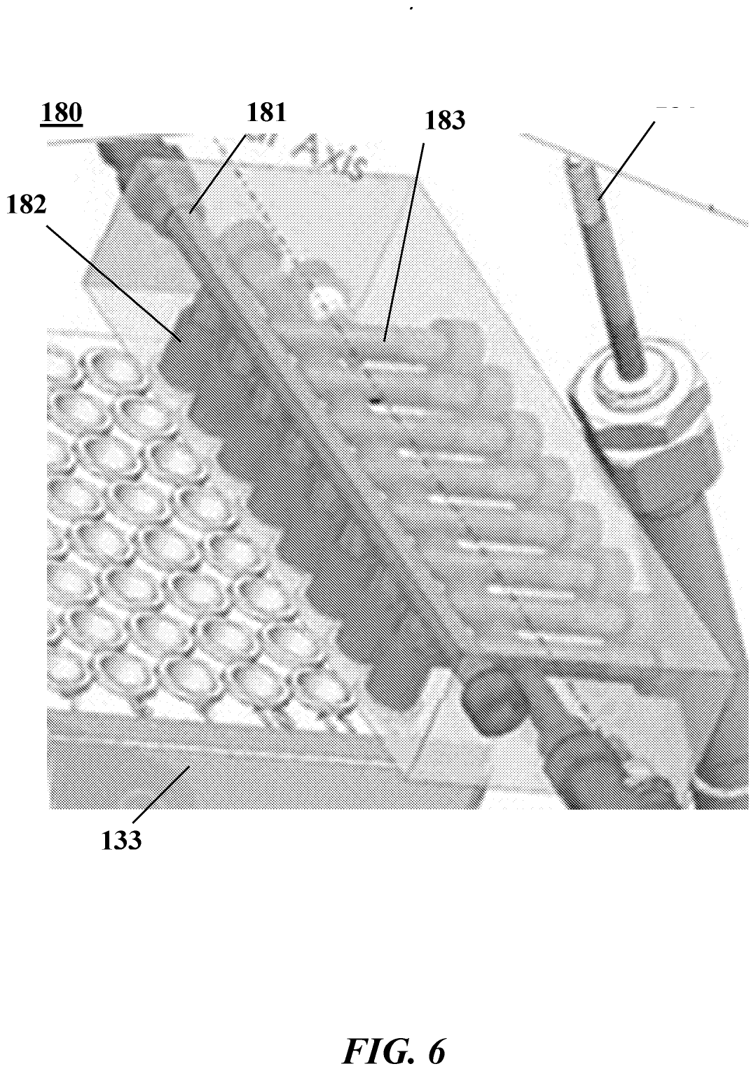

The automated robotic handling system 100 may comprise one or more vacuum bars 180. The automated robotic handling system may comprise at least about 1, 2, 3, 4, 5, 6, 7, 8, 9, 10, or more vacuum bars. The automated robotic handling systems may comprise at most about 10, 9, 8, 7, 6, 5, 4, 3, 2, or fewer vacuum bars. The one or more vacuum bars may be vacuum bars as described elsewhere herein (e.g., the vacuum bar of FIG. 6).

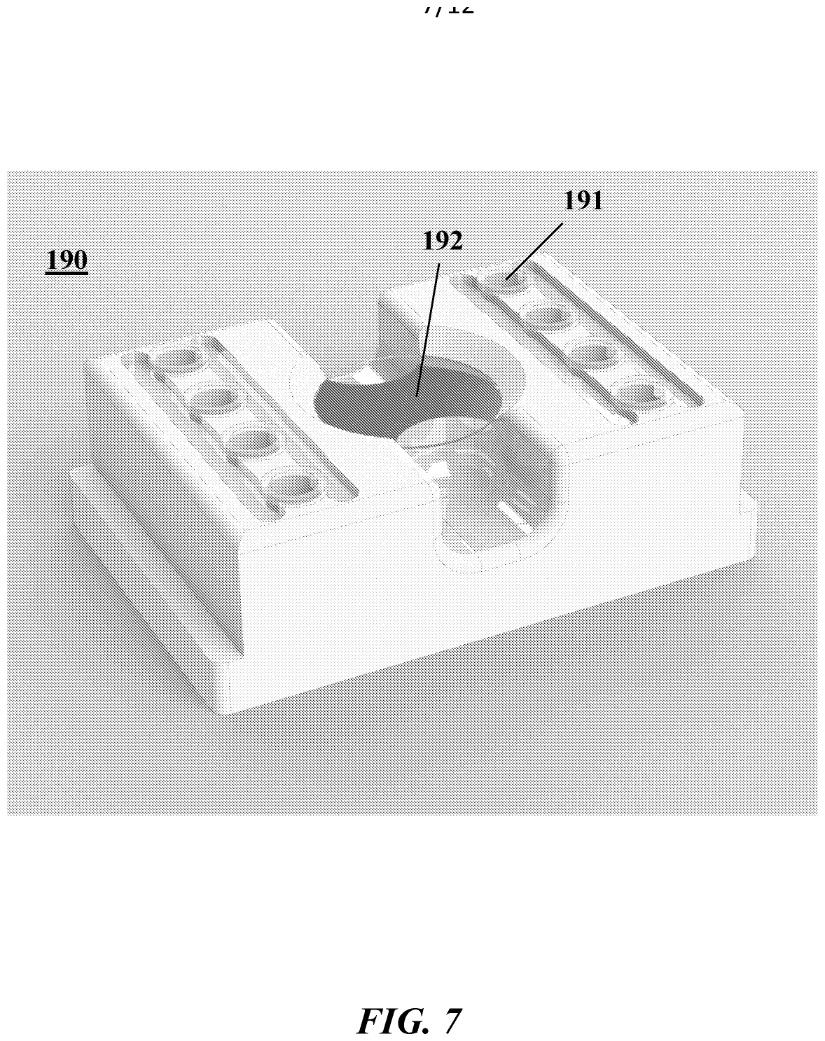

The automated robotic handling system 100 may comprise one or more output stages 190. The one or more output stages may comprise one or more output stages as described elsewhere herein (e.g., the output stage of FIG. 7). The automated robotic handling system may comprise at least about 1, 2, 3, 4, 5, 6, 7, 8, 9, 10, or more output stages. The automated robotic handling systems may comprise at most about 10, 9, 8, 7, 6, 5, 4, 3, 2, or fewer output stages.

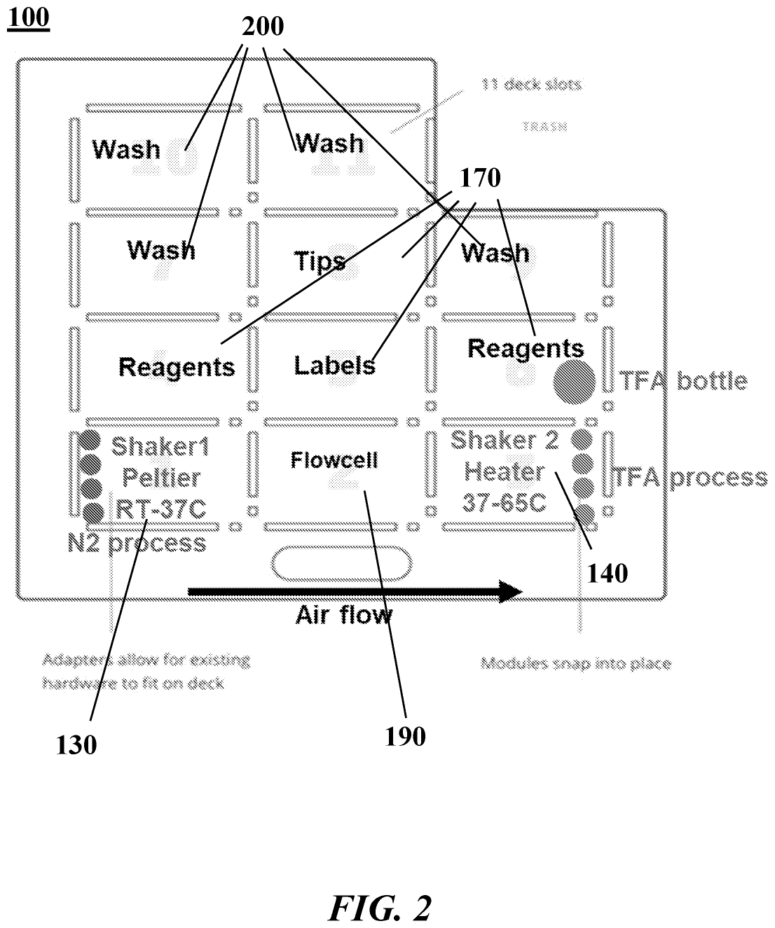

FIG. 2 shows a layout of components for an automated robotic handling system 100. FIG. 2 may be a top-down layout schematic of the components of the automated robotic handling system 100 of FIG. 1. The elements of FIG. 2 may be as described in FIG. 1. For example, the reaction stages 130 and 140 may be the same elements in FIG. 1 and FIG. 2. The one or more washes 200 may comprise one or more wash reagents as described elsewhere herein. Though shown here as comprising 4 washes, the automated robotic handling system may comprise at least about 1, 2, 3, 4, 5, 6, 7, 8, 9, 10, or more washes. The automated robotic handling systems may comprise at most about 10, 9, 8, 7, 6, 5, 4, 3, 2, or fewer washes.

FIGS. 3A-3B provide a sectional view of a lantern bar 160 and a full view of a lantern bar 160, respectively. The lantern bar may comprise a manipulation port 161. The manipulation port may be configured to interface with an arm (e.g., arm 110 or 120). The manipulation port may facilitate movement of the lantern bar throughout the handling system. The manipulation port may have other configurations as well. For example, the manipulation port can be configured to interact with a pin-based system, a hook and loop system, a pneumatic system, or the like. The manipulation port may be comprised within a support bar 162. The support bar may be a substrate holder as described elsewhere herein. The support bar may comprise one or more of polymers, metals, alloys, natural fiber materials (e.g., cellulose based materials), or the like, or any combination thereof. The support bar may be configured to hold one or more supports 163. The one or more supports may be configured in a line (e.g., a 1×n configuration, where n is an integer), a rectangular array (e.g., an n×m configuration, where n and m are independent integers), a circular array, a different polygonal array, or the like, or any combination thereof. The lantern bar may comprise at least about 1, 2, 3, 4, 5, 6, 7, 8, 9, 10, 11, 12, 13, 14, 15, 16, 17, 18, 19, 20, 25, 30, 32, 35, 40, 45, 48, 50, 60, 70, 80, 90, 96, 100, 150, 192, 200, 250, 300, 350, 384, 400, 500, 600, 700, 800, 900, 1,000, 1,250, 1,500, 1,536, or more supports. The lantern bar may comprise at most about 1,536, 1,500, 1,250, 1,000, 900, 800, 700, 600, 500, 400, 384, 350, 300, 250, 200, 192, 150, 100, 96, 90, 80, 70, 60, 50, 48, 45, 40, 35, 32, 30, 25, 20, 19, 18, 17, 16, 15, 14, 13, 13, 11, 10, 9, 8, 7, 6, 5, 4, 3, 2, or fewer supports. The supports may comprise one or more of polymers, metals, alloys, natural fiber materials, or the like, or any combination thereof,

The supports 163 may be configured to interface with a lantern substrate 164. The lantern substrate may be a lantern as described elsewhere herein. Each support may be connected to a substrate. For example, an array of 8 supports can be coupled to 8 different substrates. Examples of substrates include, but are not limited to, alkyl polymers (e.g., polyethylene, polypropylene, etc.), fluoropolymers (e.g., Teflon-AF (Dupont), Cytop® (Asahi Glass, Japan)), aromatic polymers (e.g., polyxylenes (Parylene, Kisco, Calif), polystyrene, polymethmethylacrytate), metal surfaces (e.g., gold coating)), substrates subjected to coating schemes (e.g., spin-coating, dip-coating, electron beam deposition for metals, thermal vapor deposition and plasma enhanced chemical vapor deposition), substrates subjected to functionalization methodologies (e.g., polyallylamine grafting, use of ammonia gas in PECVD, doping of long chain end-functionalized fluoroalkanes etc.), or the like, or any combination thereof. Additional details about lantern substrates can be found in Example 2. The lantern substrate may be configured for use in transporting a sample through the handling system. For example, the lantern substrate can be functionalized to bind an analyte in a sample, which can then be transported between wells of a plate in the handling system. In this example, the analyte can be disassociated from the substrate upon movement from one well to another. The lantern substrate may be functionalized with a biomolecule. For example, a biomolecule can be bound to the lantern substrate. The lantern substrate may be configured to bind to the biomolecule via one or more linkers.

The lantern bar 160 may comprise one or more guides 165. The guides may be configured to decrease movement of the lantern substrates when the lantern bar is inserted into position. The guides may be configured to ensure proper alignment of the lantern substrates within a plate well. The guides may comprise the same materials as the support bar 162. The guides may comprise a different material from the support bar 162.

FIG. 4 illustrates a shaker element 130. The shaker element may be configured for use in a sample preparation process as described elsewhere herein. Though described as a shaker element, the shaker element may not move during operation of the automated robotic handling system. For example, the shaker element can remain still during sample processing. The automated robotic handling system may comprise at least about 1, 2, 3, 4, 5, 6, 7, 8, 9, 10, or more shaker elements 130. The automated robotic handling systems may comprise at most about 10, 9, 8, 7, 6, 5, 4, 3, 2, or fewer shaker elements 130.

The shaker element 130 may comprise a base 131. The base may be configured to anchor the shaker element to the automated robotic handling system 100. For example, the base can be configured to be affixed to the automated robotic handling system via one or more screws. In another example, the base can be configured to snap into the automated robotic handling system. In some cases, the base is configured to be movable within the automated robotic handling system. For example, the base can be on a rail such that the shaker element can be moved around the automated robotic handling system. The base may comprise one or more ports configured to enable remote operation of the shaker element. Examples of ports include, but are not limited to electrical power ports (e.g., a port configured to accept power to the shaker element, a port configured to transmit power from the shaker element, etc.), data ports (e.g., a port configured to permit communication between the shaker element and one or more computers, an RJ45 port, a universal serial bus (USB) port, a serial port, etc.), sensor ports (e.g., a port configured to accept a sensor to the shaker plate), or the like, or any combination thereof. The base may be configured to transmit from a port in the base into another element of the shaker element. For example, power can be relayed from the base into an additional element of the shaker element that uses the power.

The shaker element 130 may comprise a thermoelectric element 134 and a heat exchanger 132. The thermoelectric element may comprise one or more thermoelectric heaters and/or coolers. A single thermoelectric element may be configured to be used as both a thermoelectric heater and a thermoelectric cooler. For example, the direction of current flowing through the thermoelectric element can determine the direction of the temperature change. The thermoelectric element may be configured to heat and/or cool samples on the shaker element. The thermoelectric element may be heated and/or cooled by one or more heat transfer lines. For example, a temperature-controlled water bath can be used to maintain an efficient operating temperature for the thermoelectric element.

The thermoelectric element may be configured to maintain a sample temperature of at least about 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, or more degrees Celsius. The thermoelectric element may be configured to maintain a sample temperature of at most about 75, 70, 65, 60, 55, 50, 45, 40, 39, 38, 37, 36, 35, 34, 33, 32, 31, 30, 29, 28, 27, 26, 25, 24, 23, 22, 21, 20, 15, 10, 5, 0, or less degrees Celsius. The thermoelectric element may be configured to maintain a sample temperature of the ambient temperature of the automated robotic handling system. For example, the thermoelectric element can maintain an ambient temperature during an endothermic reaction by supplying heat to the sample. The thermoelectric element may be associated with one or more temperature sensors. The temperature sensors may provide feedback to the automated robotic handling system on the temperature of the samples. For example, the temperature sensors can generate a feedback loop that maintains the samples at a predetermined temperature.

The shaker element may comprise one or more light sources. The one or more light sources may be powered through the base. The one or more light sources may be powered by cables independent of the base. The one or more light sources may be configured to initiate, sustain, terminate, or any combination thereof a chemical reaction. For example, the one or more light sources can supply energy to initiate a radical photochemical cleavage in the sample. The one or more light sources may comprise one or more lasers (e.g., a single wavelength laser, a supercontinuum laser, etc.), incoherent light sources (e.g., a light emitting diode, an incandescent light source, etc.), or the like, or any combination thereof. The one or more light sources may comprise one or more broad spectrum light sources (e.g., one or more light sources that emit a plurality of wavelengths of light), single wavelength light sources (e.g., light sources that emit or are filtered to emit a single wavelength of light), or the like, or any combination thereof. The one or more light sources may be configured to emit light with a wavelength of at least about 200, 250, 300, 350, 400, 450, 500, 550, 600, 650, 700, 750, 800, or more nanometers. The one or more light sources may be configured to emit light with a wavelength of at most about 800, 750, 700, 650, 600, 550, 500, 450, 400, 350, 300, 250, 200, or less nanometers. The one or more light sources may be configured to emit light with a wavelength as defined by any two of the proceeding values. For example, the one or more light sources can be configured to emit light with a wavelength range of about 350 to 450 nanometers.

The heat exchanger may be configured to remove excess heat from a thermoelectric element and/or a light within the shaker. The heat exchanger may be configured to transfer heat from the air around the shaker into the shaker to offset excess cold from a thermoelectric element. The heat exchanger may comprise a metal heat exchanger (e.g., copper, aluminum, etc.), a polymer heat exchanger, a liquid heat exchanger, a graphite heat exchanger, or the like, or any combination thereof. The heat exchanger may be cooled by one or more fans 135. A fan can be configured to circulate air around the heat exchanger to aid in the dissipation of heat or cold from the device.

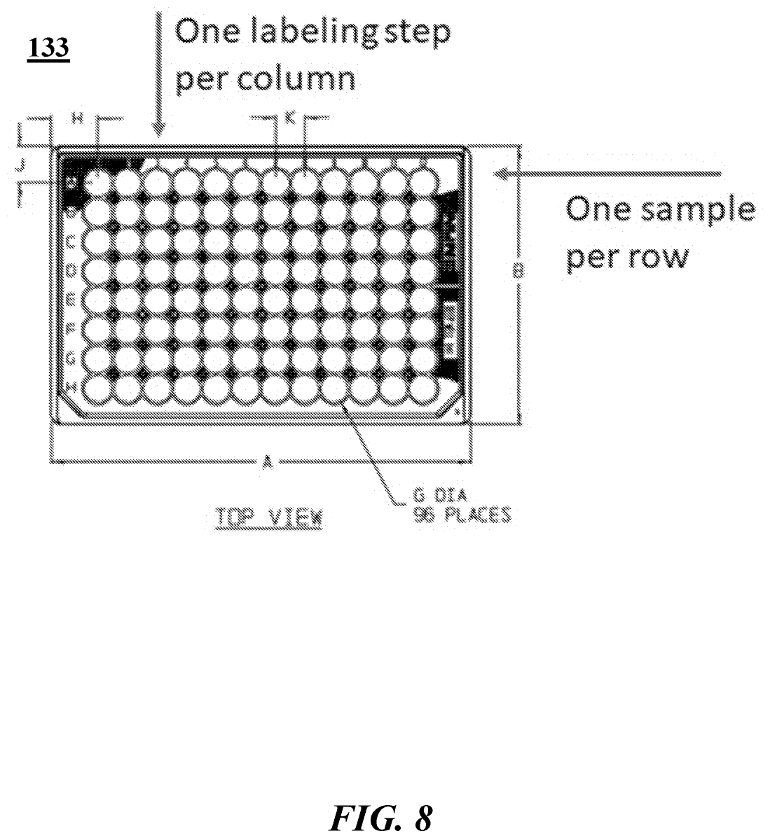

The shaker element may comprise a well plate 133. The well plate may be a well plate as described in FIG. 8. The well plate may be configured to contain one or more samples prior to, during, and/or after processing. The well plate may comprise at least about 1, 2, 3, 4, 5, 6, 7, 8, 9, 10, 11, 12, 13, 14, 15, 16, 17, 18, 19, 20, 25, 30, 32, 35, 40, 45, 48, 50, 60, 70, 80, 90, 96, 100, 150, 192, 200, 250, 300, 350, 384, 400, 500, 600, 700, 800, 900, 1,000, 1,250, 1,500, 1,536, or more wells. The well plate may comprise at most about 1,536, 1,500, 1,250, 1,000, 900, 800, 700, 600, 500, 400, 384, 350, 300, 250, 200, 192, 150, 100, 96, 90, 80, 70, 60, 50, 48, 45, 40, 35, 151, 0, 25, 20, 19, 18, 17, 16, 15, 14, 13, 13, 11, 10, 9, 8, 7, 6, 5, 4, 3, 2, or fewer wells. The well plate may be formed of plastics (e.g., polyethylene, polypropylene, polystyrene, polytetrafluoroethylene, etc.), glasses (e.g., silicon dioxide), metals (e.g., stainless steel, iron, aluminum, etc.), or the like, or any combination thereof. The well plate may be removably affixed to the shaker element. The well plate may be permanently affixed to the shaker element. The well plate may be configured to interface with a lantern bar 160 as shown in FIG. 4. For example, the well plate can have a dimension that is the same as a dimension of the lantern bar, thus permitting the lantern bar to insert the lanterns into the wells of the plate. The lantern bar may then be used to transport analytes and samples between the wells of the plate. The shaker element may be configured to interface with a gas pump bar 150 as described elsewhere herein. The gas pump bar may be attached to a gas delivery line 136. The gas deliver line may be as described elsewhere herein (e.g., configured to permit delivery of gas to the bar and subsequently to the well plate).

FIG. 5 illustrates a combined heating and evaporation element 140. The heating and evaporation element may comprise a shaker element 130 as described in FIG. 4. For example, the shaker element may comprise a base, a thermoelectric element, and a well plate. The combined heating and evaporation element may further comprise one or more evaporation bars 180. The one or more evaporation bars may be as described in FIG. 6.

FIG. 6 illustrates a detail of an evaporation bar 180. The evaporation bar may be configured to assist in the removal of a solvent, volatile compound, or liquid from one or more wells of well plate 133. For example, the evaporation bar can be configured to remove a solvent from a sample within a well. The evaporation bar may be configured to provide a reduced pressure to the one or more wells of the well plate. For example, the evaporation bar can be configured to provide a partial vacuum to the one or more wells of the well plate. The evaporation bar may be configured to provide a vacuum of at least about 0.005, 0.01, 0.05, 0.1, 0.5, 1, 2, 3, 4, 5, 6, 7, 8, 9, 10, 25, 50, 100, 150, 200, 250, 300, 350, 400, 450, 500, 550, 600, 650, 700, 750, or more Torr. The evaporation bar may be configured to provide a vacuum of at most about 750, 700, 650, 600, 550, 500, 450, 400, 350, 300, 250, 200, 150, 100, 50, 25, 10, 9, 8, 6, 5, 4, 3, 2, 1, 0.5, 0.1, 0.05, 0.01, 0.005, or fewer Torr.

The evaporation bar may comprise one or more gas passageways 181. The gas passageway may be configured to provide a flow of gas towards or away from the well plate 133. For example, the gas passageway can be configured to remove gas from the well plate. The gas passageway may be connected to one or more ports 182. The one or more ports may be configured to couple the gas passageway to one or more wells of the well plate. For example, the ports can be configured to provide a gas tight seal to the well plate while permitting gas flow to the gas passageway. The one or more ports may comprise a seal (e.g., a polymer seal) configured to improve the coupling of the ports to a well. The evaporation bar may be configured with one or more additional ports 183. The one or more additional ports may be configured such that they interface with the wells when the evaporation bar is in a different position from where the one or more ports interface with the wells. For example, the first ports can be interfaced with the wells, the evaporation bar can be rotated 90 degrees, and the second ports can interface with the wells. The one or more second ports may be configured to impart a different condition on the wells from the ports. Examples of conditions include, but are not limited to, vacuum, increased pressure, gas flow (e.g., inert gas flow), reagent introduction, or the like. For example, the ports can be configured to apply a vacuum to the wells, while the additional ports can be configured to blow an inert gas over the wells. The evaporation bar may be connected to an arm 184. The arm may be configured to adjust the positioning of the evaporation bar. For example, the arm can be configured to rotate the evaporation bar.

FIG. 7 illustrates an output stage 190. The output stage may be configured to receive a processed sample from the automated robotic handling system 100. For example, a sample can be processed to provide a fluorophore labeled amino acid sequence, and the labeled sequence can be provided to the output stage for transfer to a fluorosequencing system. The output stage may comprise one or more polymers (e.g., plastics, polyethylene, polytetrafluoroethylene, etc.), metals (e.g., iron, steel, aluminum, zinc, etc.), alloys, natural materials, or the like, or any combination thereof. For example, the output stage can be a plastic output stage. The output stage may be formed by three-dimensional (3D) printing. The output stage may be configured to be resistant to solvents and/or solvent vapors. For example, the output stage can be resistant to alcohols and trifluoroacetic acid vapor.

The output stage may comprise one or more cells 191. The output stage may comprise at least about 1, 2, 3, 4, 5, 6, 7, 8, 9, 10, 11, 12, 13, 14, 15, 16, 17, 18, 19, 20, 25, 30, 32, 35, 40, 45, 48, 50, or more cells. The output stage may comprise at most about 50, 48, 45, 40, 35, 32, 30, 25, 20, 19, 18, 17, 16, 15, 14, 13, 13, 11, 10, 9, 8, 7, 6, 5, 4, 3, 2, or fewer cells. The cells may be configured to hold a transport container. Examples of transport containers include, but are not limited to, vials, microcentrifuge tubes, well plates, slides, or the like. The cells may be configured to hold a sample directly. For example, a processed sample can be pipetted into a cell. In this example, the output stage can be moved to a fluorosequencing system, and the processed sample can be extracted from the cell and fluorosequenced. Each cell may be associated with a cap configured to cover the cell. Each cell may be left open. The one or more cells may be interfaced with a microfluidic system. For example, each cell can be configured as an input for a fluidic or microfluidic flow system configured to move processed samples to a storage or imaging system. In this example, a sample can be inserted into the cell and carried via the microfluidic system to the storage or imaging system. The output stage may comprise a port 192. The port may be configured to interface with an automated art for movement of the output stage. For example, an output arm can lock into the port, and subsequently move the output stage to a storage area. The output stage may be configured to interface with one or more additional processing systems. Examples of additional processing systems include, but are not limited to, high-performance liquid chromatography (HPLC) systems, mass spectrometers, optical measurement instruments, lyophilizing systems, enrichment systems, digestion systems, or the like, or any combination thereof.

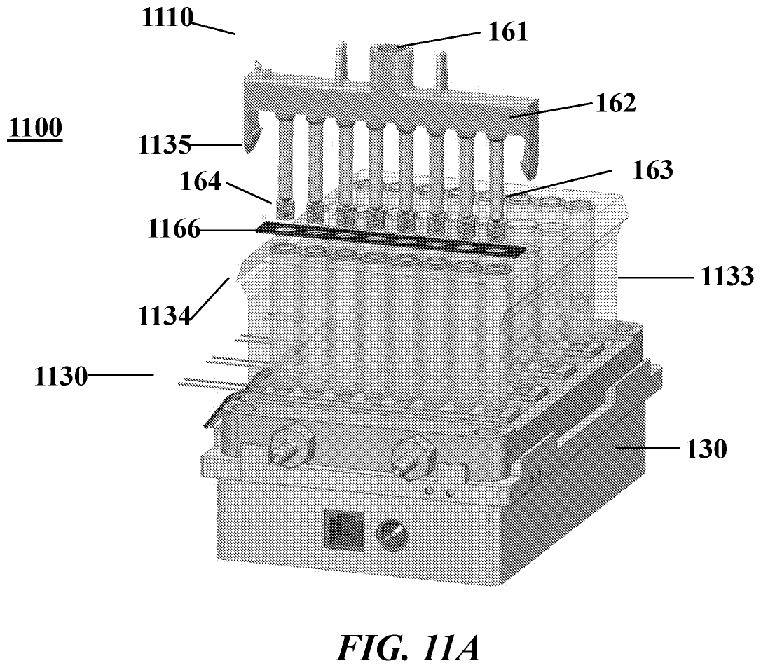

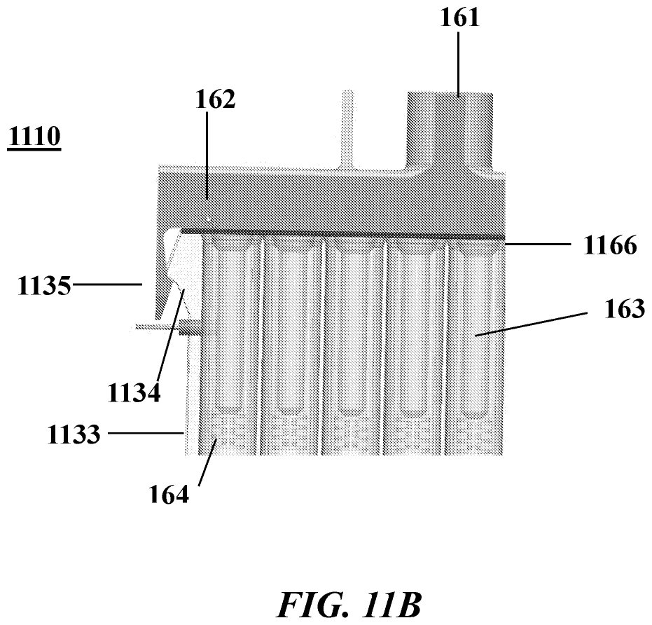

FIG. 11A provides an exploded view of a hermetically sealed lantern or N2 bar system 1100. FIG. 11B is a sectional view of a hermetically sealed lantern or N2 bar 1110. The sealed lantern or N2 bar system 1100 and hermetically sealed lantern or N2 bar 1110 may be used in some processes, such as automated peptide functionalization, which may include elevated temperatures and purging with inert gasses, which may lead to evaporation of low volumes of water or organic solvents and maintenance of inert environments. Other examples of applications of the hermetically sealed lantern or N2 bar include inert reactions including, but not limited to, photochemistry, catalysis, and environment sensitive reactions. The hermetically sealed lantern or N2 bar may be used with reactions with low volumes, as low volumes of solvent are easily evaporated, even at room temperature and solvents with low volatility. Other example applications of the hermetically sealed lantern or N2 bar include reactions with volatile organic solvents, as highly volatile solvents often require sealing, and reactions at elevated temperatures, which may require sealing to prevent evaporation at elevated temperatures. The hermetically sealed lantern or N2 bar system 1100 and hermetically sealed lantern or N2 bar 1110 may improve such processes by including a capacity to seal to prevent escape of solvent vapors and an ability to maintain an inert environment. Specifically, the hermetically sealed lantern or N2 bar 1110 reduce or eliminate evaporation of solvents in current bars. This may be accomplished by purging the hermetically sealed lantern or N2 bar 1110 with N2, then sealing the hermetically scaled lantern or N2 bar 1110; in contrast, a process with current bars, which purge the bar with N2 for the duration of the reaction, may lead to more evaporation. The hermetically sealed lantern or N2 bar 1110 may also be used for both lanterns or beads.

In some embodiments, the hermetically sealed lantern or N2 bar system 1100 includes the hermetically sealed lantern or N2 bar 1110 and a sealing shaker 1130. The hermetically sealed lantern or N2 bar 1110 may be lowered into the sealing shaker 1130 and sealed with the sealing shaker 1130. In some embodiments, the hermetically sealed lantern or N2 bar 1110 has N2 blown through it before being lowered into the sealing shaker 1130. The hermetically sealed lantern or N2 bar 1110 may then remain sealed with the sealing shaker 1130 for the remainder of a reaction time, according to some embodiments. The hermetically sealed lantern or N2 bar 1110 may also seal with the sealing shaker 1130 during the duration of a reaction to prevent evaporation according to some embodiments.

The sealing shaker 1130 may comprise a shaker element 130 (e.g., as described in FIG. 4). For example, the shaker element may comprise a base, a thermoelectric element, and a well plate. The sealing shaker 1130 may further comprise additional heating and/or cooling elements, which may be beneficial in some processes in which a sealed lantern or N2 bar may be desired, such as automated peptide functionalization, according to some embodiments. In some embodiments, the cooling element in the sealing shaker 1130 may be used to cool the hermetically sealed lantern or N2 bar 1110 to room temperature. The sealing shaker 1130 may also comprise a sealing well plate 1133, according to some embodiments. The sealing well plate 1133 may comprise a well plate 133 (e.g., as described in FIG. 4). The sealing well plate 1133 may also comprise a well plate locking mechanism 1134 to lock the looking well plate 1133 to the hermetically sealed lantern or N2 bar 1110. In some embodiments, the well plate locking mechanism 1134 may comprise a raised lip to lock with the hermetically sealed lantern or N2 bar 1110.

The hermetically sealed lantern or N2 bar 1110 may comprise a lantern bar 160 (e.g., as described in FIG. 3A), including at least a manipulation port 161, a support bar 162, supports 163, and lantern substrates 164. The hermetically sealed lantern or N2 bar 1110 also comprises a bar locking mechanism 1165, which integrates with the well plate locking mechanism 1134. In some embodiments, the bar locking mechanism 1165 comprises a clip, which covers the well plate locking mechanism 1134 to lock the hermetically sealed lantern or N2 bar 1110 in place. In some embodiments, the hermetically sealed lantern or N2 bar 1110 exerts downward pressure on the sealing well plate 1133 while locked. In some embodiments, the hermetically sealed lantern or N2 bar 1110 may also comprise a gasket 1166, which makes contact between the hermetically sealed lantern or N2 bar 1110 and the sealing well plate 1133. The gasket 1166 forms a seal between the hermetically sealed lantern or N2 bar 1110 and the sealing well plate 1133 when the hermetically sealed lantern or N2 bar 1110 is locked to the sealing well plate 1133 or downward pressure is continuously applied to the hermetically sealed lantern or N2 bar 1110 while on the sealing well plate 1133. In some embodiments, the gasket is made of Kalrez, PTFE, or a PTFE/Silicone hybrid material.

The hermetically sealed lantern or N2 bar 1110 may be either a sealed lantern bar or an N2 bar. The N2 bar 1110 comprises a hole made through the N2 bar to the attachment of the lantern substrates 164, while the hermetically sealed lantern may not. In some embodiments, the hermetically sealed lantern or N2 bar 1110 may comprise PEEK or polypropylene.

The hermetically sealed lantern or N2 bar 1110 may be capable of holding a number of lanterns 164, including at least about 1, 2, 3, 4, 5, 6, 7, 8, 9, 10, 11, 12, 13, 14, 15, 16, 17, 18, 19, 20, 25, 30, 32, 35, 40, 45, 48, 50, 60, 70, 80, 90, 96, 100, 150, 192, 200, 250, 300, 350, 384, 400, 500, 600, 700, 800, 900, 1,000, 1,250, 1,500, 1,536, or more lanterns 164. The hermetically scaled lantern or N2 bar 1110 may comprise at most about 1,536, 1,500, 1,250, 1,000, 900, 800, 700, 600, 500, 400, 384, 350, 300, 250, 200, 192, 150, 100, 96, 90, 80, 70, 60, 50, 48, 45, 40, 35, 32, 30, 25, 20, 19, 18, 17, 16, 15, 14, 13, 13, 11, 10, 9, 8, 7, 6, 5, 4, 3, 2, or fewer lanterns 164. In some embodiments, the hermetically sealed lantern or N2 bar 1110 comprises 8 lanterns 164.

The hermetically sealed lantern or N2 bar 1110 may be sealed to the sealing well plate 1133 by maintaining contact between the hermetically sealed lantern or N2 bar 1110 and the sealing well plate 1133 by the gasket 1166 and/or by tight contact between the hermetically scaled lantern or N2 bar 1110 and individual wells of the sealing well plate 1133. In some embodiments, the individual wells of the sealing well plate 1133 may comprise tapered walls, which may assist in achieving tight contact with the hermetically sealed lantern or N2 bar 1110. A weight or downward force may be applied to the hermetically sealed lantern or N2 bar 1110 to press the hermetically sealed lantern or N2 bar 1110 to the sealing well plate 1133 to achieve a seal and/or tight contact.

In some embodiments, to seal the N2 bar 1110, the N2 bar 1110 is first held over the sealing well plate 1133 and N2 is blown through the N2 bar 1110 for a duration of purging time. Once the duration of purging time is complete, the N2 bar 1110 is sealed with the sealing well plate 1133 by lowering the N2 bar 1110 into the sealing well plate 1133. In some embodiments, the N2 bar 1110 may then be locked into place with the sealing well plate 1133 by a locking mechanism such as by the sealing well plate locking mechanism 1134 locking with the bar locking mechanism 1135.

To seal the sealed lantern bar 1110, a well in the sealing well plate 1133 is loaded with reagents. The sealed lantern bar 1110 is then lowered into the wells of the sealing well plate 1133 until a tight seal is made between sealed lantern bar 1110 and the sealing well plate 1133. In some embodiments, the sealed lantern bar 1110 may then be locked into place with the sealing well plate 1133 by a locking mechanism such as by the sealing well plate locking mechanism 1134 locking with the bar locking mechanism 1135.

In some embodiments, once sealing is no longer necessary, the hermetically sealed lantern or N2 bar 1110 may first be unlocked from the sealing well plate 1133. The hermetically scaled lantern or N2 bar 1110 by be unlocked from the sealing well plate 1133 by releasing the bar locking mechanism 1135 from the well plate locking mechanism 1134. In some embodiments, the force of the pipette may be strong enough to release the hermetically sealed lantern or N2 bar 1110 without unlocking the bar locking mechanism 1135 from the well plate locking mechanism 1134.

In some embodiments, at some points during the reaction duration, an intermittent purge may be necessary in some processes. To perform an intermittent purge, the bar locking mechanism 1135 is unlocked from the well plate locking mechanism 1134, according to some embodiments. Once released, the N2 bar 1110 is lifted from the sealing well plate 1133 and purged by forcing N2 through the N2 bar 1110 for the duration of the purge. Once the duration of the purge is complete, the N2 bar 1110 is lowered into the sealing well plate 1133 and a downward weight or force is applied to the N2 bar 1110 to seal the N2 bar 1110 with the scaling well plate 1133. In some embodiments, the N2 bar 1110 is locked to the sealing well plate 1133 by engaging the bar locking mechanism 1135 with the sealing well locking mechanism 1134.

### Fluorosequencing

Various aspects of the present disclosure provide compositions and methods for peptide fluorosequencing. A fluorosequencing method disclosed herein can provide peptide sequence information at the single molecule level. In an example, fluorosequencing methods may be described in U.S. Pat. No. 9,625,469, U.S. Patent Publication No. US20200124613A1, and U.S. Pat. No. 10,545,153, each of which is incorporated by reference herein in its entirety). A method of the present disclosure may subject a peptide to fluorosequencing and an additional form of analysis. For example, a molecule of hemoglobin may be interrogated for glycation with immunostaining, and then subsequently digested and subjected to fluorosequencing for sequencing analysis.

A characteristic feature of many fluorosequencing methods is coupling amino acid labels to a peptide to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels. A fluorosequencing method may comprise labeling one, two, three, four, five, six, or more different types of amino acids residues in a subject peptide or protein. A plurality of amino acid residues may include, for example, an N-terminal amino acid, cysteine, lysine, glutamic acid, aspartic acid, tryptophan, tyrosine, serine, threonine, arginine, histidine, methionine, or any combination thereof. Each of these amino acid residues may be labeled with a different labeling moiety. Multiple amino acid residues may be labeled with the same labeling moiety such as (i) aspartic acid and glutamic acid or (ii) serine and threonine.

A label may comprise a detectable moiety. The detectable moiety may be optically detectable (e.g., fluorescent, phosphorescent, luminescent, or light absorbing). The detectable moiety may be electrochemically detectable (e.g., a redox active moiety with a characteristic oxidation or reduction potential). The detectable moiety may comprise a mass tag (e.g., for identification with mass spectrometry. A detectable moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different detectable moiety that uniquely identifies the label by its type.

A label may lack a detectable moiety. A detectable moiety-free label may be used to block an amino acid or amino acid type during a labeling operation, thereby preventing one or more types of amino acids from reacting with a label. For example, a method may comprise coupling a label to cysteine residues before providing a label with specificity for cysteine and lysine, thereby preventing the label from coupling to cysteine residues present in a system.

A label (e.g., a detectable moiety-free label) may reversibly or irreversibly bind to an amino acid type, and thus may be chemically (e.g., by addition of a cleavage reagent) or physically (e.g., by addition of heat or light) decoupled from a target peptide. A method may thus comprise blocking a first amino acid type (e.g., coupling a detection moiety-free label to cysteine), labeling a second amino acid type (e.g., threonine), unblocking the first amino acid type (e.g., decoupling a label from cysteine), and labeling the first amino acid type. Examples of reversible labels include can include silanes (e.g., trimethylsilane), acetyl groups, benzoyl groups, unsaturated pyran and furan groups, urea-forming groups, carbamate-forming groups, carbonate-forming groups, thiourea-forming groups, thiocarbamate-forming groups, thiocarbonate-forming groups, and derivatives thereof. Examples of irreversible labels can include alkyl groups, oxo-groups, amide-forming groups (e.g., an acyl chloride configured to convert an amine into an amide), and derivatives thereof.

Labeling specificity can be a major challenge for a fluorosequencing method. In many cases, a label may comprise reactivity toward a plurality of amino acid types. For example, some maleimide labels can react with cysteine, lysine, and N-terminal amines. A number of strategies may be employed to utilize or prevent such cross-reactivity. A method may comprise sequential amino acid labeling, for example to ensure that a multi-specific label is added to a system after one or more amino acid types with which the multi-specific label is configured to couple are chemically blocked or labeled, and therefore unable to react with the multi-specific label.

Discriminating between comparably reactive amino acid residues can require precise ordering of labeling operations. In the above maleimide example, lysine may be discriminated from cysteine by first reacting cysteine with a cysteine specific labeling operation (e.g., blocking cysteine in an iodoacetamide coupling operation performed at pH 7-8), thereby preventing further cysteine labeling in a subsequent lysine labeling operation. A method may comprise cysteine labeling prior to lysine labeling. A method may comprise cysteine labeling prior to glutamate labeling. A method may comprise cysteine labeling prior to aspartate labeling. A method may comprise cysteine labeling prior to tryptophan labeling. A method may comprise cysteine labeling prior to tyrosine labeling. A method may comprise cysteine labeling prior to serine labeling. A method may comprise cysteine labeling prior to threonine labeling. A method may comprise cysteine labeling prior to histidine labeling. A method may comprise cysteine labeling prior to arginine labeling. A method may comprise lysine labeling prior to glutamate labeling. A method may comprise lysine labeling prior to aspartate labeling. A method may comprise lysine labeling prior to tryptophan labeling. A method may comprise lysine labeling prior to tyrosine labeling. A method may comprise lysine labeling prior to serine labeling. A method may comprise lysine labeling prior to threonine labeling. A method may comprise lysine labeling prior to arginine labeling. A method may comprise carboxylate side chain (e.g., glutamate and aspartate side chain) labeling prior to tryptophan labeling. A method may comprise carboxylate side chain (e.g., glutamate and aspartate side chain) labeling prior to tyrosine labeling. A method may comprise carboxylate side chain (e.g., glutamate and aspartate side chain) labeling prior to serine labeling. A method may comprise carboxylate side chain (e.g., glutamate and aspartate side chain) labeling prior to threonine labeling. A method may comprise carboxylate side chain (e.g., glutamate and aspartate side chain) labeling prior to histidine labeling. A method may comprise carboxylate side chain (e.g., glutamate and aspartate side chain) labeling prior to arginine labeling. A method may comprise at least 2, at least 3, at least 4, at least 5, or at least 6 amino acid labeling operations performed in a sequence configured to minimize or prevent label cross-reactivity (e.g., labeling more than the intended type or types of amino acids).

Fluorosequencing may comprise removing peptides through techniques such as Edman degradation following or preceding subject peptide detection. Sequential peptide removal may generate sequence or position-specific information. For example, a reduction in fluorescence following an N-terminal amino acid removal operation may indicate that a labeled amino acid, and thus that a specific type of amino acid, was disposed at a peptide N-terminal. Removal of each amino acid residue can carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

A labeling moiety used in the instant application may be configured to withstand conditions for removing one or more of the amino acid residues. Some non-limiting examples of potential labeling moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5)6-naphthofluorescein. The labeling moiety may be a fluorescent peptide or protein or a quantum dot.

Peptide detection or imaging may comprise immobilizing the peptide on a surface. The peptide may be immobilized to the surface by coupling a peptide-derived cysteine residue, the peptide N terminus, or the peptide C terminus with the surface or with a reagent coupled to the surface. The peptide may be immobilized by reacting the cysteine residue with the surface or with a capture reagent coupled to the surface. The peptide may be immobilized by coupling the peptide C-terminus or N-terminus with a capture moiety described herein. The peptide may be immobilized on a surface. Detecting the immobilized peptide may comprise capturing an image comprising the peptide. The image may comprise a spatial address specific to the peptide. A plurality of peptides may be detected in a single imagine, wherein one or more of the peptides may comprise a spatial address within the image. The surface may be optically transparent across the visible spectrum and/or the infrared spectrum. The surface may possesses a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 pm thick, between 1 and 5 pm thick, between 2 and 10 pm thick, between 5 and 20 pm thick, between 20 and 50 pm thick, between 50 and 200 pm thick, between 200 and 500 pm thick, or greater than 500 pm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that may sequester peptides for sequencing and modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more labeling moieties (e.g., amino acid labels) coupled to the peptide. The sequencing technique may comprise imaging a plurality of peptides or proteins to determine the presence of one or more labeling moieties on individual peptides from among the plurality of peptides. The sequencing technique may comprise imaging at least 103, at least 104, at least 105, at least 106, at least 107, at least 108 or more proteins or peptides (e.g., imaging a portion of a surface comprising at least 103 to at least 108 proteins or peptides). These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (SEQ ID NO: 1, wherein ‘K’ denotes lysine, ‘D’ denotes aspartate, ‘Y’ denotes tyrosine, ‘A’ denotes alanine, and ‘G’ denotes glycine), and may comprise labels coupled to each lysine and tyrosine residue. A first image comprising the C-terminal immobilized peptide may indicate the presence of two lysines and one tyrosine in the peptide. The N-terminal amino acid may be removed (e.g., by Edman degradation), such that a second image comprising the C-terminal immobilized peptide may indicate the presence of one lysine and one tyrosine in the peptide. This process may be repeated until a sequence of KXXYXXXXXXXKX is identified for the peptide, wherein ‘X’ indicates anon-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and comparing these locations to known peptide sequences, which may identify the entire list of amino acid residues in the peptide sequence. For example, identifying the positions of the lysines and cysteines in a 40 amino acid fragment of a human protein may uniquely identify the protein (e.g., only one human protein contains the specific pattern of lysine and cysteine residues identified in the 40 amino acid fragment).

An imaging method may involve a variety of different spectrophotometric and microscopy methods, such as fluorimetry, diffuse reflectance, interferometric scattering, Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and imaging a subject peptide, the position of the labeled amino acid residue can be determined in the peptide.

### Selective Amino Acid Labeling

Various aspects of the present disclosure provide methods for selectively labeling types (e.g., lysine, tyrosine, or phosphotyrosine) or groups (e.g., carboxylate side chain-containing or aromatic side chain-containing) of amino acids. A composition, system, or method of the present disclosure may selectively label cysteine, lysine, tyrosine, histidine, glutamic acid, aspartic acid, tyrosine, threonine, serine, arginine, N-terminal amines, C-terminal carboxyl-groups, or any combination thereof. A composition, system, or method may selectively label a group of amino acids, for example, a substituted maleimide reagent may couple to lysine and cysteine residues present in a sample.

The free thiol group of a cysteine side often the most nucleophilic group in a peptide (Scheme 1), and thus may promiscuously react with a range of reagents. To prevent such cross-reactivity, thiol side chains are often reacted early in a labeling (e.g., a multi-labeling scheme), thereby blocking them from further reactions. An example of a thiol-selective reaction is an iodoacetamide coupling operation. Such a reaction may be performed in pH ranges which limit (e.g., prevent) lysine cross reactivity, such as at a sufficiently low pH to ensure lysine protonation, which may diminish lysine reactivity.

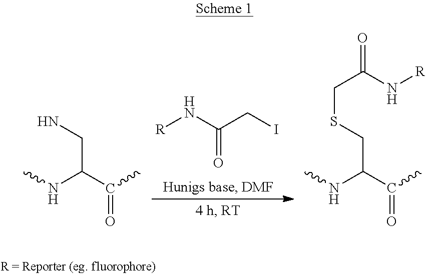

Scheme 2 provides an example of a lysine labeling reaction. The a lysyl amine (e.g., a lysyl butylamine sidechain) can be selectively labeled with an ester (e.g., an NHS ester). This operation may be performed after cysteine labeling in cases where cross-reactivity may be possible.

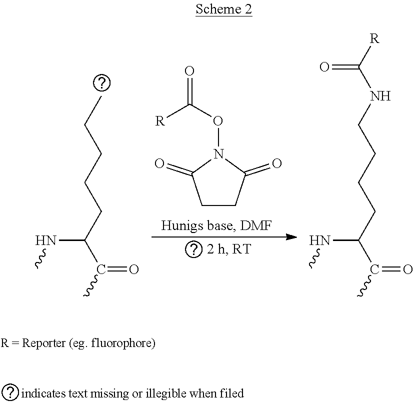

Peptide carboxylates may be labeled through amine coupling, an example of which is provided in Scheme 3. Carboxyl-side chains (e.g., those of aspartic acid and glutamic acid) and C-terminal carboxyls can be converted to amides via amine-based nucleophilic substitution. The resulting amides may comprise detectable moieties, chemically inert groups, or reactive handles for further coupling. For example, an amine reagent for carboxylate amidation may comprise an alkyne suitable for a subsequent coupling operation. In particular instances, a polypeptide is digested using GluC under pH 8 digestion buffer or a sufficiently similar protease/buffer system such that the cleavage site occurs on the C-terminal-side of an acidic residue (e.g., aspartic acid and glutamic acid). Such a digestion method can generate peptides wherein every carboxyl-residue (e.g., glutamic acid and aspartic acid) is disposed at a peptide C-terminus, thus enabling C-terminal selective amino acid immobilization. Whether the C-terminal carboxylic acid, the side chain carboxylic acid, or both are amidated and immobilized to the support may not affect the function of the systems, methods, and kits as disclosed herein. Alternate reactive groups can be used in place of an alkyne. However, for brevity, only the alkyne example is discussed above.

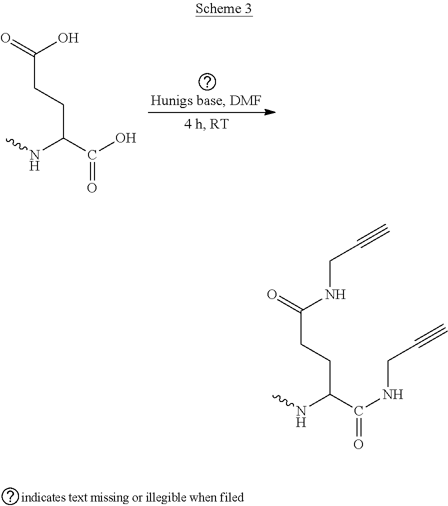

Scheme 4 provides an example of tyrosine-specific labeling. The position adjacent (e.g. ortho to) the tyrosine phenol hydroxyl carbon can be labeled through a two-operation labeling process using a bifunctional diazonium reagent. Following diazo-coupling to tyrosine, a second reagent (such as a dithiolane) may optionally be coupled to the diazo label (e.g., to selectively couple a detectable moiety to the labeled tyrosine). Alternatively, the diazonium reagent may comprise a detectable moiety or may lack chemically reactive handles for further coupling.

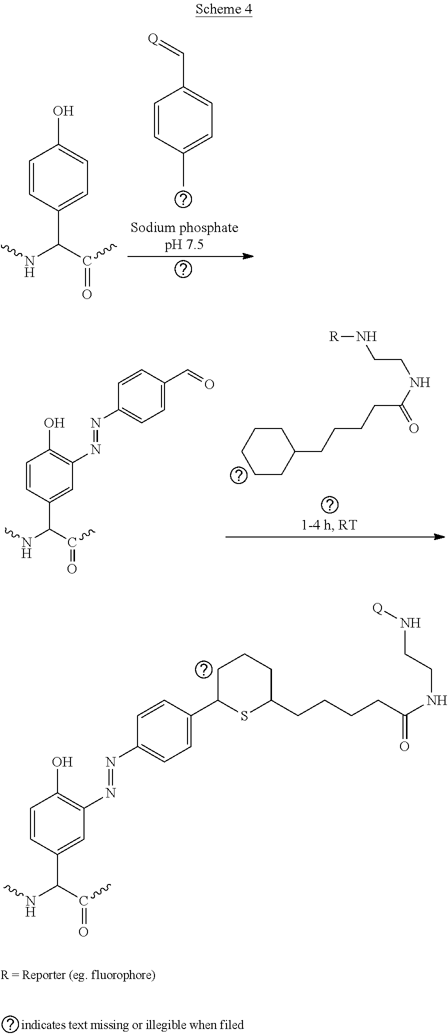

Scheme 5 provides an example of a histidine coupling scheme. A histidine imidazole nitrogen can be labeled through a two-operation labeling process using an alpha-beta unsaturated carbonyl compound, such as 2-cyclohexenone. The alpha-beta unsaturated carbonyl compound may react with histidine in a nucleophilic addition reaction. The alpha-beta unsaturated carbonyl may comprise a detectable moiety. Following histidine coupling, the alpha-beta unsaturated carbonyl may be further coupled to an additional label, such as a dithiolane. Histidine may alternatively be selectively coupled to an epoxide reagent.

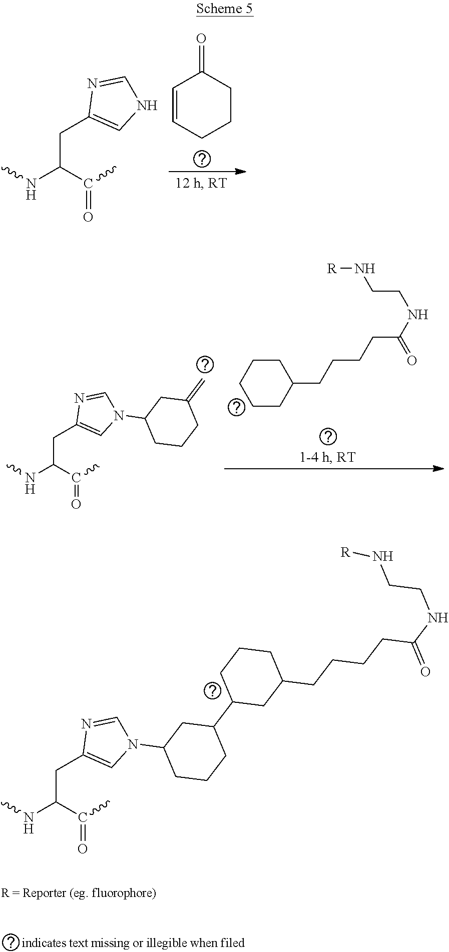

Scheme 6 provides an example of an arginine labeling mechanism. An arginine guanidinium can be acylated (e.g., labeled with an NHS ester with the aid of Barton's base). This example reaction may show cross-reactivity or interference by primary amines (e.g., N-terminus, lysine) or thiols (e.g., cysteine), and thus may be performed after N-terminal support immobilization and cysteine and lysine labeling in order to prevent or diminish cross-reactivity.

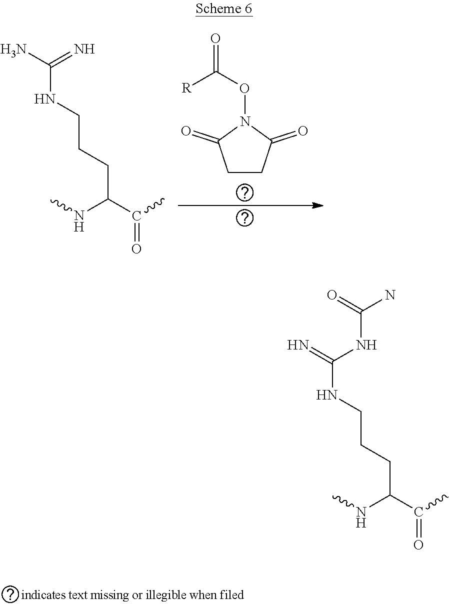

Methionine comprises a relatively low nucleophilicity and can often be selectively labeled by a redox based scheme where an oxaziridine group reacts specifically with a methionine thioether without cross-reacting with cysteine (Scheme 7). The bond formed is stable to reducing agents such as TCEP.

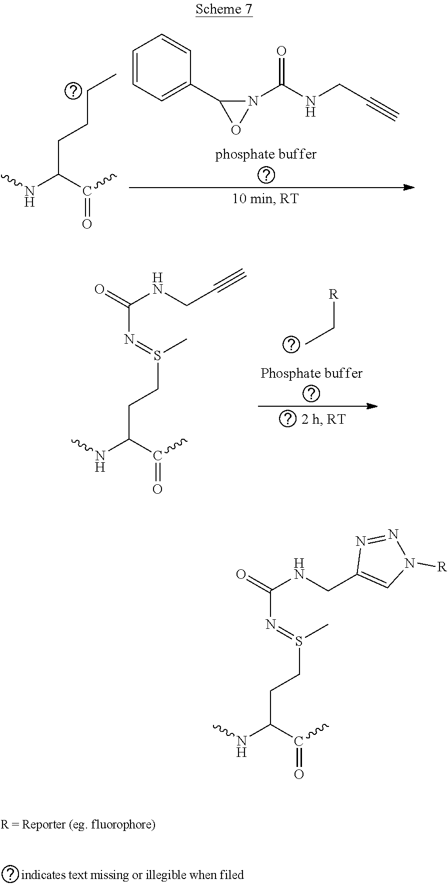

Scheme 8 provides an example of a tryptophan labeling scheme. A tryptophan indole may couple to a diazopropanoate ester, yielding a tertiary amine derivatized tryptophan, The coupling may be metal-catalyst mediated, for example by a dirhodamine(II) tetraacetate complex, which may enhance the selectivity for tryptophan over other amino acid types.

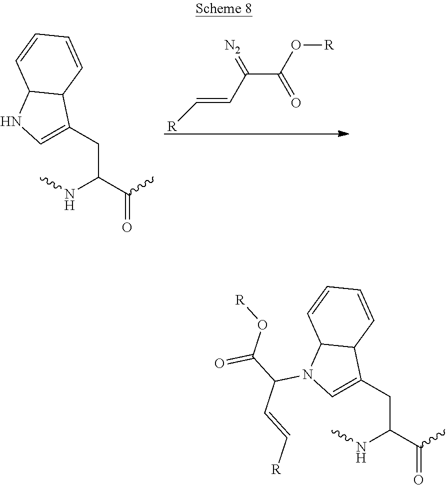

Phosphorylated amino acids such as phosphoserine, phosphotyrosine, or phosphothreonine can be selectively labeled. Such a labeling method may distinguish between types of phosphorylated amino acids. For example, Scheme 9 below provides a phosphoryl beta-elimination followed by a label conjugate addition (e.g., a Michael acceptor reaction) operation for selectively labeling of phosphoserine (pSer) and phosphothreonine (pThr) over other phosphorylated amino acids such as phosphotyrosine (pTyr). A subsequent pan-phospho labeling method can be implemented to label pTyr.

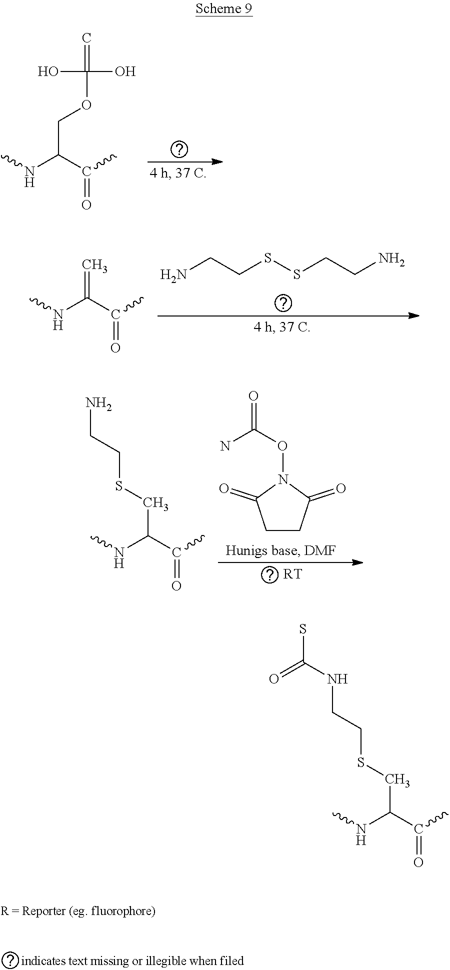

### Peptide Degradation

Chemical techniques that allow for the mild and sequential protein degradation conditions can be important for proteomics. Degradation can be used as a method to sequence polymers (e.g., proteins or peptides) to determine the order and identity of the amino acids of a polymer. A peptide or protein may be subsequently subjected to additional cleavage conditions until the sequence of at least a portion of the peptide or protein is identified. The entire sequence of a peptide or a protein may be determined using the methods and compositions described herein. Removal of each amino acid residue may be carried out through a variety of techniques including, for example, Edman degradation, organophosphate degradation, or proteolytic cleavage. In some aspects, Edman degradation may be used to remove a terminal amino acid residue. These terminal amino acid residues may be removed from either the C-terminus or the N-terminus of the peptide chain. In some instances, the amino acid residue at the N-terminus of the peptide chain may be removed. A chemical or enzymatic technique for removing a terminal amino acid may remove a defined number of (e.g., exactly one) amino acid. Accordingly, a method for analyzing a peptide may comprise successive degradation and analysis operations, such that the removal of a defined number of amino acids from an N-terminus or C-terminus per operation provides position and sequence specific amino acid identifications during analysis. A chemical or enzymatic technique for removing a terminal amino acid may cleave a peptide at a defined location (e.g., only in between two alanine residues).

An Edman degradation method may comprise chemically functionalizing a peptide N-terminus or C-terminus (e.g., to form a thiourea or a guanidinium derivative of an N-terminal amine), and then contacting the functionalized terminal amino acid with a reagent (e.g., a hydrazine), a condition (e.g., a high or low pH or temperature), or an enzyme (e.g., an Edmanase with specificity for the functionalized terminal amino acid) to remove the functionalized terminal amino acid.

A diactivated phosphate or phosphonate may be used for peptide cleavage. Such a method may utilize an acid to remove a functionalized amino acid. The diactivated phosphate or phosphonate may be a dihalophosphate ester. In other embodiments, the techniques involve using an enzyme to remove the terminal amino acid residue, such as, for example, an exopeptidase or an Edmanase. For example, a method may comprise derivatizing an N-terminal amino acid of a peptide with a diactivated phosphate, and contacting the peptide with an Edmanase with cleavage activity toward phosphate-functionalized N-terminal amino acids.

Peptide cleavage conditions may be achieved with a solvent. The solvent may be an aqueous solvent, organic solvent, or a combination thereof. The solvent may be a mixture of solvents. The solvent may be an organic solvent. The organic solvent may be anhydrous. The solvent may be anon-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or apolar protic solvent (e.g., isopropanol (IPA), ethanol, methanol, acetic acid, water, etc.). The solvent may be a polar aprotic solvent. The solvent may be DMF. The solvent may be a Ci-Ci2haloalkane. The Ci-Ci2haloalkane may be DCM. The solvent may be a mixture of two or more solvents. The mixture of two or more solvents may be a mixture of a polar aprotic solvent and a Ci-Cmhaloalkane. The mixture of two or more solvents may be a mixture of DMF and DCM. The mixture of solvents may be any combination thereof.

A degradation process may comprise a plurality of operations. For example, a method may comprise an initial operation for derivatizing a terminal amino acid of a peptide, and a subsequent operation for cleaving the derivatized terminal amino acid from the peptide. One such method comprises organophosphorus compound-mediated N-terminal functionalization and removal, and thus provides an alternative to the isothiocyanate (e.g., phenyl isothiocyanate) based processes of some Edman degradation schemes.

An organophosphate-based degradation scheme may comprise dissolving the peptide in an organic solvent or organic solvent mixture (e.g., a mixture of dichloromethane and dimethylformamide) in the presence of an organic base (e.g., triethylamine, N, N-diisopropylethylamine (DIPEA), 1,8-diazabicyclo[5.4.0]undec-7-ene (DBU), pyridine, 1,5-diazabicyclo(4.3.0)non-5-ene, 2,6-di-tert-butylpyridine, imidazole, histidine, sodium carbonate, etc.). The peptide may then be contacted with at least one organophosphorus compound. The cleavage of the peptide or protein N-terminus may be initiated through the addition of a weak acid (e.g., formic acid in water). The cleavage of the peptide or protein N-terminus may also be initiated with water. The resulting products may include the terminal amino acid of the peptide or protein released from the peptide as a phosphoramide and the peptide or protein that is shortened by the terminal amino acid residue, which comprises a free N-terminus that can be used to perform a subsequent cleavage reaction.

The reaction mixture may comprise a stoichiometric or an excess concentration of the cleavage compound (e.g., relative to the concentration of peptides to be cleaved). The reaction mixture may comprise at least about 0.001% v/v, about 0.01% v/v, about 0.1% v/v, about 1% v/v, about 5% v/v, about 10% v/v, about 15% v/v, about 20% v/v, about 30% v/v, about 40% v/v, about 50% v/v, or more of the cleavage compound. The reaction mixture may comprise at most about 50% v/v, about 40% v/v, about 30% v/v, about 20% v/v, about 15% v/v, about 10% v/v, about 5% v/v, about 1% v/v, about 0.1% v/v, about 0.01% v/v, about 0.001% v/v, or less of the cleavage compound. The reaction mixture may comprise from about 0.1% v/v to about 20% v/v, about 0.5% v/v to about 10% v/v, or about 1% v/v to about 10% v/v of the cleavage compound. The reaction mixture may comprise about 5% v/v of the cleavage compound.

The reaction may be performed at a temperature of at least about 0° C., about 5° C., about 10° C., about 15° C., about 20° C., about 25° C., about 30° C., about 40° C., about 50° C., about 60° C., about 70° C., or more. The reaction may be performed at a temperature of at most about 70° C., about 60° C., about 50° C., about 40° C., about 30° C., about 25° C., about 20° C., about 15° C., about 10° C., about 5° C., about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature.

The peptide and the cleavage compound may be mixed or incubated for at least about 1 minute, about 5 minutes, about 10 minutes, about 20 minutes, about 30 minutes, about 40 minutes, about 50 minutes, about 60 minutes, about 2 hours, about 3 hours, about 4 hours, about 6 hours, about 8 hours, about 10 hours, about 12 hours, about 16 hours, about 20 hours, about 24 hours, or more. The peptide and the cleavage compound may be mixed or incubated for at most about 24 hours, about 20 hours, about 16 hours, about 12 hours, about 10 hours, about 8 hours, about 6 hours, about 4 hours, about 3 hours, about 2 hours, about 1 hour, about 50 minutes, about 40 minutes, about 30 minutes, about 20 minutes, about 10 minutes, about 5 minutes, about 1 minute, or less. The peptide and the cleavage compound may be mixed or incubated from about 1 minute to about 24 hours, 5 minutes to about 6 hours, 5 minutes to about 2 hours, or 5 minutes to about 30 minutes.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition of synthetic peptides. A sample may comprise a single species or a mixture of species. A biological sample may comprise biomaterial from a single organism, from a colony of genetically near-identical organisms, or from multiple organisms (e.g., enterocytes and microbiota from a human digestive tract). A biological sample may be fractionated (e.g., plasma separated from whole blood), filtered, or depleted (e.g., high abundance proteins such as albumin and ceruloplasmin removed from plasma).

A sample may comprise all or a subset of the biomolecules from the subject, tissue sample, cell culture, cell, or portion thereof. For example, a sample from a subject may comprise the majority of proteins present in that subject, or may comprise a small subset of the proteins from that subject. A biological sample may comprise a bodily fluid such as cerebral spinal fluid, saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which it was sourced) or purified, such that the macromolecule comprises at least 0.5%, at least 1%, at least 2%, at least 3%, at least 4%, at least 5%, at least 7.5%, at least 10%, at least 15%, at least 20%, at least 25%, at least 30%, at least 40%, at least 50%, at least 60%, at least 70%, at least 75%, at least 80%, at least 90%, at least 95%, at least 98%, or at least 99% of a composition by weight (e.g., by dry weight or including solvent). The biological sample may be complex, and may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (peptides, nucleic acids, lipids, small molecules). A biological sample may comprise a plurality of peptides configured for a method of the present disclosure (e.g., digestion, C-terminal labeling, or fluorosequencing).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and buffycoat proteins may be partitioned and separately analyzed) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides from the resulting lysate, and analyzing the peptides by a fluorosequencing method of the present disclosure. A method may comprise capturing peptides from a sample using a C-terminal capture reagent, and analyzing the peptides (e.g., by a fluorosequencing method).

Methods consistent with the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and the subject of the second analytical method may be derived from the same subject or the same sample. For example, a method may comprise collecting cell free DNA and a peptides from a human plasma sample, sequencing the cell free DNA (e.g., to identify a cancer marker), and performing proteomic analysis on the plasma proteins.

Labels with Multiple Reactive Groups

Aspects of the present disclosure provide amino acid labels comprising a first reactive group for coupling to an amino acid (or a portion thereof, such as a reactive functional group of an amino acid side chain) and a second reactive group for coupling to a reporter moiety or a protecting group. Such a system may be referred to as a “click-clack” labeling system, wherein a “click” reagent refers to a label configured to couple to an amino acid, and a “clack” reagent refers to a reporter moiety or protecting group configured to couple to the “click” reagent. The second reactive group of a label may be configured to reversibly or irreversibly couple to a reporter moiety, a protecting group, or any combination thereof. The second reactive group may be reversibly coupled to a protecting group, decoupled from the protecting group, and then coupled to a reporter moiety. For example, the label may be provided with a protecting group coupled to its first or second reactive group (e.g., a diol coupled to an aldehyde reactive group of the label). Such a modular labeling process may enable multi-amino acid labeling schemes with diminished cross-reactivity between amino acid and label types. Such a labeling process may also enable the use of chemically sensitive reporter moieties (e.g., pH sensitive or chemically quenchable dyes), by allowing their attachment following amino acid labeling operations. For example, a method may comprise selectively labeling cysteine residues of a peptide with a first label, selectively labeling lysine residues of the peptide with a second label, selectively labeling carboxylate-containing residues (e.g., aspartate and glutamate) of the peptide with a third label, selectively labeling arginine residues of the peptide with a fourth label, chemically modifying (e.g., oxidizing) methionine residues of the peptide, selectively labeling the chemically modified methionine residues of the peptide with a fifth label, and coupling different reporter moieties (e.g., different color dyes) to each of the first, second, third, fourth, and fifth labels in a single operation (e.g., upon addition of all labeling reagents simultaneously). It is also possible that one or more reporter fluorophores may directly label the amino acids on the peptide chains. A bifunctional label of the present disclosure may prevent cross-reactivity between a first reactive group of a label and a reporter moiety. For example, the use of bifunctional labels may permit use of reporter moieties which are cross-reactive with a first reactive group of a label, such as an iodoacetamide-reactive dye and a label comprising a cysteine reactive iodoacetamide group.

A label of the present disclosure may be used to crosslink two biological species, such as two amino acid residues. For example, a method may comprise coupling a lysine selective label to a first peptide and a cysteine selective label to a second peptide, and then cross-linking the lysine and cysteine selective labels. The cross-linking may directly couple (e.g., through a chemical bond) the lysine and cysteine selective labels, or may comprise a linker, such as a “clack” reagent configured to couple to second reactive groups on the lysine and cysteine selective labels.

Examples of amino acid selective labels comprising second reactive groups, as well as example reagent pairs for their syntheses, are provided in TABLE 1. A cysteine- and lysine-selective “Click” label may comprise an iodoacetamide as a first reactive group (e.g., for coupling to cysteine or lysine) and an azide as a second reactive group (e.g., for coupling to a “Clack” reporter moiety or protecting group), such as the iodoacetamide PEG azide compound shown in Row A of TABLE 1. A cysteine-selective “Click” label may comprise an iodoacetamide as a first reactive group and a norbomene as a second reactive group, such as the reactant shown in Row B of TABLE 1. Such a reagent may be synthesized by coupling a norbomene amine with an iodoacetamide N-hydroxysuccinamide ester. A cysteine-selective “Click” label may comprise an iodoacetamide as a first reactive group and an aldehyde as a second reactive group, such as 2-iodo-N-(3-oxopropyl) acetamide (as shown in Row C of TABLE 1). Such a compound may be generated by coupling an N-hydroxysuccinamide ester with an amine comprising a geminal diether configured to hydrolyze to an aldehyde. A cysteine-selective label may comprise a first reactive group for coupling to cysteine but lack a second reactive group (e.g., the label may be a “dummy” label), and therefore be unable to couple to a “Clack” reporter moiety or protecting group) reagent. An example of such a reagent may be iodoacetamide, as shown in TABLE 1 Row D.

A lysine-selective “Click” label may comprise an N-hydroxysuccinamide ester as a first reactive group and a norbomene as a second reactive group, such as the reagent shown in Row F of TABLE 1. A lysine-selective “Click” label may comprise an N-hydroxysuccinamide ester as a first reactive group and a geminal diether as a second reactive group, such as the reagent shown in Row G of TABLE 1. Such a reagent may be generated by coupling 1-hydroxypyrrolidine-2,5-dione to the carboxylic acid of a compound comprising a geminal diether. A lysine-selective label may comprise a first reactive group for coupling to lysine but lack a second reactive group for coupling to a “Clack” reporter moiety or protecting group. An example of such a reagent may be an activated ester, such as the compound shown in Row H of TABLE 1

A carboxylate-selective (e.g., selective for aspartate and glutamate side chain carboxylates) “Click” label may comprise an amine as a first reactive group and an azide as a second reactive group, such as the reagent shown in Row I of TABLE 1. A carboxylate-selective “Click” label may comprise an amine as a first reactive group a norbomene as a second reactive group, such as the reagent shown in Row J of TABLE 1. A carboxylate-selective “Click” label may comprise an amine as a first reactive group a geminal diether as a second reactive group such as the reagent shown in Rows K and L of TABLE 1. A carboxylate-selective label may comprise a first reactive group for coupling to a carboxylate but lack a second reactive group for coupling to a “Clack” reporter moiety or protecting group. An example of such a reagent may be an alkyl amine, such as the compound shown in Row M of TABLE 1.

A phosphoserine-, phosphothreonine-, and/or glycosylation-selective “Click” reagent may comprise a disulfide as a first reactive group and an azide, a norbomene, a geminal diether, or an aldehyde as a second reactive group, as shown in Rows N-R of TABLE 1. A phosphoserine-, phosphothreonine-, and/or glycosylation-selective “Click” reagent may comprise a disulfide as a first reactive group and may lack a second reactive group.

TABLE 1
Exemplary “Click” Labels Consistent With The Present Disclosure

| | | | COMMERCIAL |
|---|---|---|---|
| | | | AVAILABILITY & |
| “CLICK” | | | REAGENTS |
| LABEL | | | FOR “CLICK” |
| TYPE | ROW | “CLICK” LABEL | LABEL SYNTHESIS |
| CYSTEINE “CLICK” LABELS | A | 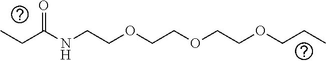 [SMILES: CCCOCCOCCOCCNC(=O)CC] | COMMERCIALLY AVAILABLE |
| | B | 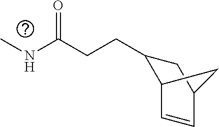 | 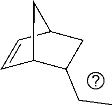 |
| | | | 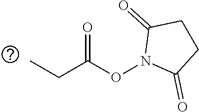 |
| | C | 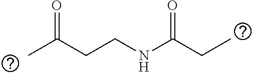 | 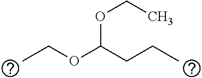 |
| | | | 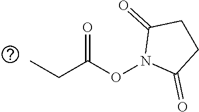 |
| | D | 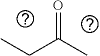 | COMMERCIALLY AVAILABLE |
| LYSINE “CLICK” LABELS | E | 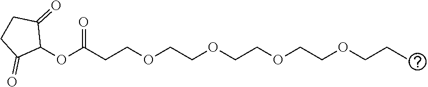 [SMILES: CCCOCCOCCOCCOCCC(=O)OC1C(=O)CCC1=O] | COMMERCIALLY AVAILABLE |
| | F | 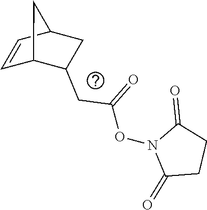 [SMILES: O=C(CC1CC2C=CC1C2)ON1C(=O)CCC1=O] | COMMERCIALLY AVAILABLE |
| | G | 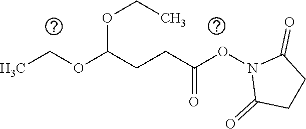 [SMILES: CCOC(CCC(=O)ON1C(=O)CCC1=O)OCC] | 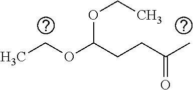 |
| | | | 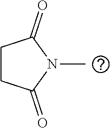 |
| | H | 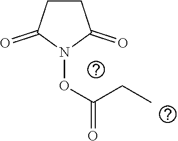 | COMMERCIALLY AVAILABLE |
| CARBOXYLATE “CLICK” LABELS | I | 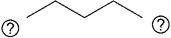 | COMMERCIALLY AVAILABLE |
| | J | 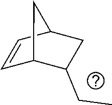 | COMMERCIALLY AVAILABLE |
| | K | 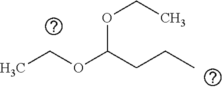 | COMMERCIALLY AVAILABLE |
| | L | | COMMERCIALLY AVAILABLE |
| | M | 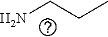 | COMMERCIALLY AVAILABLE |
| PHOSPHOSERINE, PHOSPHOTHREONINE [symbol] & GLYCOSYLATION “CLICK” | N | 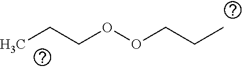 | COMMERCIALLY AVAILABLE |
| | O | 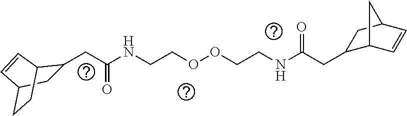 [SMILES: O=C(CC1CC2C=CC1CC2)NCCOOCCNC(=O)CC1CC2C=CC1C2] | 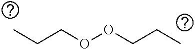 |
| | | | 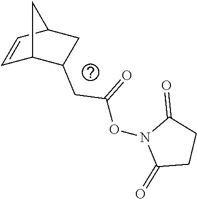 [SMILES: O=C(CC1CC2C=CC1C2)ON1C(=O)CCC1=O] |
| | P | 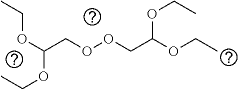 [SMILES: CCOC(COOCC(OCC)OCC)OCC] | COMMERCIALLY AVAILABLE |
| | Q | 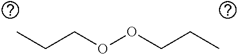 | COMMERCIALLY AVAILABLE |
| | R | | COMMERCIALLY AVAILABLE |

[symbol]

indicates data missing or illegible when filed

Sample preparation may be improved by labeling a plurality of amino acid residues through series of sequential operations. The present disclosure provides a range of systems to facilitate labeling of multiple amino types. The system may minimize cross-reactivity of amino acids, reporter moieties (e.g., fluorescent molecules (e.g., dyes)), or the decomposition of, for example, sensitive reporter moieties (e.g., fluorescent molecules (e.g., dyes)).

### Computer Systems

The present disclosure provides computer systems that are programmed to implement methods of the disclosure. FIG. 9 shows a computer system 901 that is programmed or otherwise configured to, for example, control the systems or execute the methods of the present disclosure. The computer system 901 can regulate various aspects of the present disclosure, such as, for example, the operation of an automated robotic handling system. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, status of an automated robotic handling system or a processing reaction. Examples of Ui's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, be configured to execute an automated sample processing process.

The following examples are illustrative of certain systems and methods described herein and are not intended to be limiting.

### Example 1—Use of a Well Plate in Sample Processing

Using methods and systems of the present disclosure, a well plate is used in sample processing. FIG. 8 illustrates a well plate 133. The well plate may be as described elsewhere herein. The well plate may be configured such that each row of the well plate is used for a different sample, while each column of the well plate is used for a different processing operation.

In this example, a sample can be loaded into the first row of a 96 well plate, while different samples are loaded into the other 7 rows of the plate. Each column can then be loaded with different reagents for different operations of a sample processing method. For example, the columns can be loaded with reagents configured to isolate an analyte of interest, wash the analyte, fluorescently label the analyte, and isolate the labelled analyte. Once isolated, the analyte can be transferred to a substrate or flow cell for additional processing. The analyte can be moved between wells of the plate using a lantern substrate. In this example, a lantern bar with 8 substrates can be used to move all 8 of the samples at once, improving processing speed and reducing complexity.

An example to illustrate the use of an automated robotic handling system may be as follows. The sample comprises a peptide mixture comprising two peptides, each containing amino acids-Lysines and glutamic acid which can be labeled with distinct fluorophores. The sequence of the peptides are Peptide-1 (GKAGEAGYRA; SEQ ID NO: 2) and Peptide-2 (GAKAGEAGYRA; SEQ ID NO: 3). The input sample comprises 0.2 umol of the two peptides as a mixture (Samples M1, M2), 0.2 umol of the peptide-1 (Samples A1, A2) and peptide-2 (Samples B1, B2), and negative controls-water (N1, N2), all dissolved in lOO uL water. All operations of liquid transfers and peripheral actions (such as shaker motion, LED on, etc.) are driven automatically by software unless noted. The operations of the reaction are as follows:

1. Sample and plate loading: The samples M1, M2, A1, A2, B1, B2 and N1, N2 are loaded in the first column A of the reaction plate by a user, using handheld pipettes. The following plates are placed at different locations on the robot deck (e.g., a robot deck as described in FIG. 2): 1—reaction plate, 2—2 mL 9 mm glass vial, 3—evaporation plate, 4—caddy-left, 5—reagent plate comprising one or more labels, 6—reagent bottles, 7—washplate1, 9—washplate2, 8—tiprack comprising a plurality of pipette tips, 10—washplate3, 11—washplate4. The default trash location is set at 12. The reagent plate is contained in the kit and comprises reagents (e.g., labelling reagents, wash reagents, etc.) at designated locations for the reactions to be carried out. Lanterns are placed in their default positions—N2 bar (e.g., gas pump bar as described elsewhere herein) at reaction plate, column 2 and lantern bar at washplate1, column 12. The liquids in the caddy and their positions have been defined in the kit and the software. The caddies may be reagent bottles as described elsewhere herein comprising the reagents described elsewhere herein.

2. Instrument initiation and protocol definition: The user inputs the (a) location of the samples and (b) labeling choice—e.g., lysine to be labeled with JF549 dye and glutamic acid with Atto647N dye to the software interface. The protocol is then defined computationally to perform lysine labeling using click1 (NHS-Norbomene) and glutamic acid with click2 (Amine-Azide). The corresponding fluorophores are clack1 (JF549-mTET (methyltetrazine)) and clack2 (Atto647N-DBCO).

3. C-terminal chemistry: The reagents for the photoredox chemistry (in reagent plate in column 1) are solubilized with formic acid buffer (pH 3.5, 100 mM). The solution is transferred to the destination wells (column 1 in reaction plate) containing the samples. The N2 bar is moved from its default location (reaction plate, column 2) to the 1^{st} column on the reaction plate. N2 gas is bubbled for 5 mins followed by turning on an LED light. N2 bubbling is triggered every 20 mins for 30 seconds each time. The heat exchanger is turned on and temperature set to 25° C. The shaker is turned on at 100 rpm. This set of conditions are maintained for 3 h after which the N2, LED, shaker, and temperature controller are turned off.

4. N-terminal capture on lanterns: The N2 bar is moved to its default location. HEPES buffer (pH 8.5, 1M) is transferred from reagent well (location=B1) to the LED wells in the reaction plate. The lanterns on the lantern bar are moved from the default location to the LED row (column 1 on reaction plate). The temperature is set to 37° C. and the shaker is turned on. The conditions are maintained for 16 h after which the shaker is turned off and homed. The peptides are now captured on the lantern bar.

5. Wash operation: Wash solvents (e.g., dimethylformamide, acetonitrile, acetonitrile/water (1:1 vv)+0.1% formic acid, acetonitrile/water: 1:1 vv), present in the bottles in the caddy are dispensed into the different wash plates 1-4 respectively—500 pL in each well. The lantern bar is moved from acetonitrile/water, acetonitrile/water/formic acid, acetonitrile, and finally to dimethylformamide in separate operations. Each operation involves dunking the lantern into the solution to completely immerse the lantern and moving the lantern up and down 10 times. The lanterns are then placed in the solution for 5 mins before transferred to the next wash well. The lanterns are then moved to its home location (washplate1, column 12).

6. Lysine labeling: The aliquoted reagent—40 pmole of NHS-Norbomene reagent (5-norbomene-2-acetic acid succinimidyl ester; CAS #1234203-45-2 in reagent plate, location=C1) is solubilized in 400 pL DMF (solvent in caddyright, location=A1). 50 pL of the reagent is dispensed into the 8 wells in reaction plate (location=column 3). 50 pL of DIPEA (N,N-Diisopropylethylamine) stock (reagent plate, location=B4) are dispensed into the reaction plate (column 3). 100 pL of DMF is dispensed into each well of the reaction plate in column 3. Final volume of each well is 200 pL. The sample bar is moved from its home location to the column 3. The shaker is turned on and the reaction conditions maintained for 2 h (37° C.). The shaker is turned off and homed after the operation. The lysines on the peptides are now labeled with Norbomene.

7. Wash operation: The same series of washes are performed as described in operation 5.

8. Glutamic acid labeling: The aliquoted reagent-40 pmole of Amine-Azide (3-Azidopropylamine, CAS #88192-19-2) in reagent plate, location=DI) is solubilized in 400 pL DMF. 50 pL of this reagent is transferred to each well in column 4 on the reaction plate. HCTU (O-(1H-6-Chlorobenzotriazole-1-yl)-1,1,3,3-tetramethyluroniumhexafluorophosphate, CAS #330645-87-9) is dissolved in 400 pL DMF and 50 pL of it is transferred to each well. The third reagent is DIPEA and 50 pL of it is transferred to each wells in the reaction plate. 50 pL of DMF is dispensed to the wells making a total of 200 pL. The lantern bar is moved from home location to column 4 on the reaction plate. The shaker is turned on and incubated for 2 h at 37° C. The shaker is then turned off. The glutamic acid residues on the peptides are now labeled with azide.

9. Wash operation: The sample bar is moved through the series of wash solvents as described in operation 5.

10. Fluorophore labeling (Click-clack reaction): 0.5 pmole of JF549-mTET in the wells (reagent plate, column 10) and 0.5 pmole of Atto647N-DBCO in wells (reagent plate, column 11) are each solubilized with Methanol/Water (1:1 vv) and transferred to wells in reaction plate (column 5). The sample bar is now moved from its home location to column 5 on reaction plate. The reaction conditions are setup 37° C. and shaker turned on for 12 h. This reaction leads to the labeling of lysines and glutamic acid with JF549 and Atto647N respectively.

11. Wash operation: The sample bar is moved again through the wash operations to remove excess dyes.

12. TFA cleavage operation: 190 pL of Trifluoroacetic acid is dispensed into each wells in column 12 of evaporation plate. 10 pL of water and 10 pL of Triisopropylsilane (reagent plate, location=G1) is added to the wells. The washed lantern bar is moved from home location to the TFA dispensed location. The shaker is turned on for 1 h at 25° C. This operation cleaves the fluorescently labeled peptides from the lanterns. The sample bar is now returned to its home position. Other acidic buffers with pH of 1 to 4 may be substituted for TFA, such as formic acid or glycine hydrochloride. For example, the sample bar may be incubated in the buffer for 16 hours at 60 degrees C.

13. Evaporation of TFA: The trifluoroacetic acid is evaporated using the evaporation bar. N2 gas is flown and regulated at 10 psi to remove the volatile TFA which is captured by a carbon filter. The evaporation bar is heated to 60° C. which heats the incoming N2 gas. This operation removes TFA from the wells.

14. Deprotection of N-termini of peptides: The N-termini of the liberated peptides have a protected chemical of pyridinecarboxaldehyde variant. The dhydrazine (Dimethylaminoethylhydrazine, CAS #57659-80-0 in rows in column 6, reagent plate) is dissolved in 200 pL sodium phosphate buffer (pH 7.5, 100 mM) and added to the fluorescent peptides in the wells. The temperature of the evaporation shaker is heated to 65° C. for 16 h. The reaction is then stopped and the plate cooled to room temperature. This operation is optional, and may be avoided if glycine HCl buffer is used, which deprotects the N-termini of peptide directly from the lanterns.

15. Transfer of fluorescent peptides to vials: The peptides from each well are independently transferred into the glass vials (in location 3 on deck).

### Example 2—Use of a Lantern

Using methods and systems of the present disclosure, peptides are selectively captured using a lantern. The present disclosure provides a range of substrates for capturing peptides. One such type of substrate is a lantern, which may comprise a solid support comprising peptide capture agents, and a rod for positioning the solid support within a sample. A lantern rod may be manipulatable by a user (e.g., the user may hold the lantern rod) or an instrument. A lantern solid support may comprise a reactive group of the present disclosure, such as a reactive group selective for cysteine or a peptide C-terminus.

A peptide mixture containing angiotensin (provided as a positive control peptide), a peptide comprising the sequence AKGAGRY{PRA}N-ONH2 (SEQ ID NO: 4, where {PRA} denotes Propargylglycine), a capture negative control peptide, and a peptide of interest are dissolved in approximately 500 pL of a solution comprising water, 3% acetonitrile, and 0.1% formic acid. A lantern with a solid support comprising peptide capture agents is placed in the sample and incubated for 24 hours at 37° C., providing sufficient time for angiotensin, 2K peptide, the capture negative control peptide, and the peptide of interest to couple to the peptide capture agents of the lantern. The lantern is then washed twice for two minutes in fresh deionized water to remove unbound peptides. The lantern is then dried in air or with an N2 flow. Subsequently, the lantern is placed in a clean centrifuge tube for storage or shipping. The peptides coupled to the lantern may be recovered by resuspending the lantern in a solution and providing a cleavage agent to decouple the peptides from the peptide capture agent.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for processing a biomolecule, comprising:
(a) providing, at a first location, (i) a substrate and (ii) a substrate holder coupled to said substrate, wherein said substrate comprises said biomolecule coupled thereto;
(b) automatically directing said substrate holder and said substrate from said first location to a second location different from said first location; and
(c) at said second location, processing said biomolecule to provide a processed biomolecule coupled to said substrate; wherein said processing comprises labelling said biomolecule to provide a labelled biomolecule.

2. The method of claim 1, wherein said method for processing said biomolecule is completely automated by a computer processor.

3. The method of claim 1, wherein said substrate is coupled to said substrate holder at a first position of said substrate holder, and wherein said substrate holder is coupled to an additional substrate comprising an additional biomolecule at a second position of said substrate holder.

4. The method of claim 1, wherein said substrate is coupled to said substrate holder at a first position of said substrate holder, and wherein said substrate holder is coupled to a plurality of additional substrates comprising a plurality of additional biomolecules at a plurality of positions of said substrate holder.

5. The method of claim 4, wherein said plurality of substrates comprises at least 8 substrates.

6. The method of claim 1, further comprising, coupling said biomolecule to said substrate.

7. The method of claim 6, wherein said coupling occurs at an additional location different from said first location or said second location.

8. The method of claim 6, wherein said coupling occurs at said first location.

9. The method of claim 6, wherein said biomolecule comprises a protein or a peptide.

10. The method of claim 1, wherein said substrate is a bead or a lantern.

11. The method of claim 1, wherein said substrate is a solid-support.

12. The method of claim 1, wherein (b) is performed using a robotic system configured to direct said substrate holder from said first location to said second location.

13. The method of claim 12, wherein said robotic system further comprises a fluid handling unit, a moveable stage, an evaporation unit, a vacuum, a gas pump, a light source, a temperature control unit, a shaker, a fan, or a combination thereof.

14. The method of claim 13, wherein said robotic system comprises said fluid handling unit, and wherein said method further comprises, prior to (b), providing a well and using said fluid handling unit to provide reagents into said well.

15. The method of claim 14, wherein said reagents comprise labelling agents.

16. The method of claim 15, wherein said labelling agents are fluorescent labelling agents.

17. The method of claim 15, wherein said labelling agents are configured to label one or more amino acids of a peptide.

18. The method of claim 17, wherein said labelling agents comprise a labelling agent configured to label a lysine residue, cysteine residue, glutamic acid residue, aspartic acid residue, tyrosine residue, arginine residue, histidine residue, threonine residue, serine residue, proline residue, glutamine residue, or tryptophan residue.

19. The method of claim 17, wherein said labelling agents are configured to label post-translational modifications such as phosphorylation, glycosylation, ubiquitination, or methylation.

20. The method of claim 15, wherein said processing of (c) comprises contacting said substrate coupled to said biomolecule with said reagents in said well to provide said labelled biomolecule.

21. The method of claim 20, wherein said robotic system further comprises said shaker, and wherein (c) further comprises, using said shaker to mix said reagents in said well.

22. The method of claim 20, wherein said robotic system further comprises said light source.

23. The method of claim 22, wherein (c) further comprises using said light source to attach one or more reagents of said reagents to said biomolecule, thereby providing said labelled biomolecule.

24. The method of claim 14, wherein said robotic system comprises said moveable stage, and wherein said method further comprises, using said moveable stage to move said well relative to said fluid handling unit.

25. The method of claim 13, wherein said robotic system comprises said evaporation unit, wherein said processing of (c) comprises use of a solvent, and wherein said method further comprises, subsequent to (c), using said evaporation unit to evaporate said solvent from said labelled biomolecule.

26. The method of claim 25, wherein said evaporation unit comprises said vacuum.

27. The method of claim 25, wherein said evaporation unit comprises said gas pump.

28. The method of claim 27, wherein said gas pump is coupled to a nitrogen gas stream.

29. The method of claim 13, wherein said robotic system comprises said temperature control unit, and where (c) is performed at a controlled temperature.

30. The method of claim 13, wherein said robotic system comprises a feedback mechanism that regulates said fluid handling unit, said moveable stage, said evaporation unit, said vacuum, said gas pump, said light source, said temperature control unit, said shaker, said fan, or a combination thereof.

31. The method of claim 12, wherein said robotic system accommodates a multi well plate.

32. The method of claim 1, further comprising, providing a light source at said second location.

33. The method of claim 32, wherein said light source is an LED light source.

34. The method of claim 32, wherein said light source is configured to illuminate in a pre-specified pattern.

35. The method of claim 32, wherein said light source is configured to couple to a multiwell plate and illuminate one or more wells of said multiwell plate.

36. The method of claim 32, wherein (c) comprises using said light source to conduct a photoreaction to provide said labelled biomolecule.

37. The method of claim 36, wherein said photoreaction comprises decarboxylative alkylation.

38. The method of claim 1, wherein said biomolecule comprises a peptide, and wherein said labelling comprises coupling a label to an amino acid of said peptide.

39. The method of claim 38, wherein said label comprises (i) a first reactive group that is configured to couple to a second reactive group that is coupled to a reporter moiety configured to emit a signal or (ii) a protecting group configured to prevent coupling between said label and said second reactive group.

40. The method of claim 1, further comprising, removing said labelled biomolecule from said substrate.

41. The method of claim 40, wherein said removing comprises contacting said substrate with an acidic buffer.

42. The method of claim 41, wherein said acidic buffer comprises formic acid, trifluoroacetic acid (TFA), or glycine hydrochloride.

43. The method of claim 41, further comprising, using an evaporation unit to remove said acidic buffer from said labelled biomolecule.

44. The method of claim 40, further comprising, storing said labelled biomolecule.

45. The method of claim 40, further comprising, evaporating said removed biomolecule.

46. The method of claim 45, wherein said evaporating is performed using an evaporation unit.

47. The method of claim 46, wherein said evaporation unit is coupled to a gas source or a vacuum.

48. The method of claim 47, wherein said gas source is a nitrogen stream.

49. The method of claim 1, further comprising, subjecting said labelled biomolecule to sequencing.

50. The method of claim 49, wherein said labelled biomolecule is a labelled peptide or protein and said sequencing comprises protein sequencing.

51. The method of claim 50, wherein said protein sequencing comprises fluorosequencing.

52. The method of claim 49, further comprising, prior to said sequencing, coupling said labelled biomolecule to a flow cell.

53. The method of claim 52, wherein said flow cell comprises a linker configured to couple to said labelled biomolecule.

54. The method of claim 53, wherein said flow cell comprises a set of linkers, each of said set of linkers configured to couple to one of a set of biomolecules.

55. The method of claim 1, wherein said substrate comprises a peptide capture reagent.

56. The method of claim 55, wherein said peptide capture reagent comprises a cleavable linker.

57. The method of claim 55, wherein said peptide capture reagent comprises an N-terminal capture reagent.

58. The method of claim 57, wherein said N-terminal capture reagent is pyridinecarbaldehyde (PCA) or a derivative thereof.

59. The method of claim 1, wherein said substrate comprises a poly-ethylene glycol (PEG) linker, polyacrylate, polyamide, or polystyrene.

60. The method of claim 1, wherein said biomolecule is a protein, a peptide, a lipid, a carbohydrate, a metabolite, a nucleic acid molecule, or a combination thereof.

61. The method of claim 1, wherein said biomolecule is obtained from a biological sample.

62. The method of claim 61, wherein said biological sample is a cell or tissue sample.

63. The method of claim 61, wherein said biological sample is a blood sample.

64. A system for processing a biomolecule, comprising:
a substrate comprising said biomolecule coupled thereto;
a substrate holder configured to couple to said substrate;
an automated robotic handling system configured to automatically direct said substrate holder from a first location to a second location; and
reagents for labelling said biomolecule.

65. The system of claim 64, wherein said substrate is a bead or a lantern.

66. The system of claim 64, wherein said substrate is coupled to said substrate holder at a first position of said substrate holder, and wherein said substrate holder is coupled to an additional substrate comprising an additional biomolecule at a second position of said substrate holder.

67. The system of claim 64, further comprising, a light source at said second location.

68. The system of claim 67, wherein said light source is an LED light source.

69. The system of claim 67, wherein said light source is configured to illuminate in a pre-specified pattern.

70. The system of claim 67, wherein said light source is configured to couple to a multiwell plate and illuminate one or more wells of said multiwell plate.

71. The system of claim 64, wherein said substrate is coupled to said substrate holder at a first position of said substrate holder, and wherein said substrate holder is coupled to a plurality of additional substrates comprising a plurality of additional biomolecules at a plurality of positions of said substrate holder.

72. The system of claim 71, wherein said plurality of substrates comprises at least 8 substrates.

73. The system of claim 64, further comprising a robotic system configured to direct said substrate holder from a first location to a second location.

74. The system of claim 73, wherein said robotic system further comprises a fluid handling unit, a moveable stage, an evaporation unit, a vacuum, a gas pump, a light source, a temperature control unit, a shaker, a fan, or a combination thereof.

75. The system of claim 74, wherein said robotic system comprises a feedback mechanism that is configured to regulate said fluid handling unit, said moveable stage, said evaporation unit, said vacuum, said gas pump, said light source, said temperature control unit, said shaker, said fan, or a combination thereof.

76. The system of claim 7373, wherein said robotic system comprises an evaporation unit configured to evaporate a solvent.

77. The system of claim 76, wherein said evaporation unit comprises a vacuum.

78. The system of claim 76, wherein said evaporation unit comprises a gas pump.

79. The system of claim 78, wherein said gas pump is coupled to a nitrogen gas stream.

80. The system of claim 73, wherein said robotic system is configured to accommodate a multi well plate.

81. The system of claim 64, wherein said reagents for labelling comprise labelling agents configured to label one or more amino acids of a peptide.

82. The system of claim 81, wherein said labelling agents comprise a labelling agent configured to label a lysine residue, cysteine residue, glutamic acid residue, aspartic acid residue, tyrosine residue, arginine residue, histidine residue, threonine residue, serine residue, proline residue, glutamine residue, or tryptophan residue.

83. The system of claim 81, wherein the labelling agents are configured to label post-translational modifications such as phosphorylation, glycosylation, ubiquitination, or methylation.

84. The system of claim 81, wherein said labelling agents comprise agents for performing click chemistry.

85. The system of claim 81, wherein said labelling agents comprise (i) a first reactive group that is configured to couple to a second reactive group that is coupled to a reporter moiety configured to emit a signal or (ii) a protecting group configured to prevent coupling between said label and said second reactive group.

86. The system of claim 64, wherein said biomolecule comprises a peptide.

87. The system of claim 64, further comprising reagents for removing said labelled biomolecule from said substrate.

88. The system of claim 87, wherein said reagents comprise an acidic buffer.

89. The system of claim 88, wherein said acidic buffer comprises formic acid, trifluoroacetic acid (TFA), or glycine hydrochloride.

90. The system of claim 88, further comprising, a storage unit for storing said labelled biomolecule.

91. The system of claim 64, further comprising, a flow cell configured to couple to a labelled biomolecule.

92. The system of claim 91, wherein said labelled biomolecule is a labelled peptide or protein.

93. The system of claim 91, wherein said flow cell comprises a linker configured to couple to said labelled biomolecule.

94. The system of claim 93, wherein said flow cell comprises a set of linkers, each of said set of linkers configured to couple to one of a set of biomolecules.

95. The system of claim 64, wherein said substrate comprises a peptide capture reagent.

96. The system of claim 95, wherein said peptide capture reagent comprises a cleavable linker.

97. The system of claim 95, wherein said a lipeptide capture reagent comprises an N-terminal capture reagent.

98. The system of claim 97, wherein said N-terminal capture reagent is pyridinecarbaldehyde (PCA) or a derivative thereof.

99. The system of claim 64, wherein said substrate comprises a poly-ethylene glycol (PEG) linker, polyacrylate, polyamide, or polystyrene.

100. The system of claim 64, wherein said biomolecule is obtained from a biological sample.

101. The system of claim 100, wherein said biological sample is a cell or tissue sample.

102. The system of claim 100, wherein said biological sample is a blood sample.

103. A system for processing a biomolecule, comprising:
a substrate holder configured to couple to a substrate comprising said biomolecule coupled thereto;
a robotic arm coupled to said substrate holder;
at least one computer processor configured to perform executable instructions and a memory comprising said executable instructions, which, when executed by said at least one computer processor, causes said at least one computer processor to implement a method comprising:
automatically instructing said robotic arm to direct said substrate holder and said substrate from a first location to a second location different from said first location,
wherein, at said second location, said biomolecule is labelled to provide a labelled biomolecule coupled to said substrate.

104. The system of claim 103, further comprising a fluid handling unit.

105. The system of claim 104, wherein said method further comprises instructing said fluid handling unit to provide reagents in a vessel in said second location.

106. The system of claim 105, wherein said reagents comprise labelling agents.

107. The system of claim 103, further comprising a moveable stage.

108. The system of claim 107, wherein said method further comprises instructing said moveable stage to move from said first location to said second location or from said second location to a third location different from said second location.

109. The system of claim 103, further comprising an evaporation unit.

110. The system of claim 109, wherein said method further comprises instructing said robotic arm to move said evaporation unit.

111. The system of claim 110, wherein said evaporation unit is coupled to a vacuum or a gas pump.

112. The system of claim 103, further comprising a light source.

113. The system of claim 112, wherein said method further comprises electronically turning on or off said light source or modulating an intensity thereof.

114. The system of claim 103, further comprising a temperature control unit.

115. The system of claim 114, wherein said method further comprises, controlling said temperature control unit to obtain a temperature within a range of temperatures.

116. The system of claim 115, wherein said temperature control unit comprises a fan or Peltier component.
